SEQUENCE LISTING (36 sequences)

SEQ ID NO: 1 - protein, 14 aa, synthetic construct
  REGION 1..14: note = SARS-CoV-2 3CLpro Substrate
KTSAVLQSGFRKME

SEQ ID NO: 2 - protein, 306 aa, synthetic construct [sequence omitted]

SEQ ID NO: 3 - protein, 50 aa, synthetic construct
  REGION 1..50: note = SARS-CoV-2 3CLpro
SMQNCVLKLKVDTANPKTPKYKFVRIQPGQTFSVLACYNGSPSGVYQCAM

SEQ ID NO: 4 - protein, 607 aa, synthetic construct
  REGION 1..607: note = Native Bovine Serum Albumin (BSA) protein
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 5 - protein, 74 aa, synthetic construct
  REGION 1..74: note = Myoglobin AntibodyS binding domain
GHHEAEIKPLAQSHATKHKIPVKYLEFISECIIQVLQSKHPGDFGADAQGAMNKALELFRKDMASNYKELGFQG

SEQ ID NO: 6 - protein, 238 aa, synthetic construct
  REGION 1..238: note = GFP
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 7 - protein, 110 aa, synthetic construct
  REGION 1..110: note = Human TMPRSS2
GWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 8 - protein, 306 aa, synthetic construct
  REGION 1..306: note = MERS-CoV 3CLpro
SGLVKMSHPSGDVEACMVQVTCGSMTLNGLWLDNTVWCPRHVMCPADQLSDPNYDALLISMTNHSFSVQKHIGAPANLRVVGHAMQGTLLKLTVDVANPSTPAYTFTTVKPGAAFSVLACYNGRPTGTFTVVMRPNYTIKGSFLCGSCGSVGYTKEGSVINFCYMHQMELANGTHTGSAFDGTMYGAFMDKQVHQVQLTDKYCSVNVVAWLYAAILNGCAWFVKPNRTSVVSFNEWALANQFTEFVGTQSVDMLAVKTGVAIEQLLYAIQQLYTGFQGKQILGSTMLEDEFTPEDVNMQIMGVVMQ

SEQ ID NO: 9 - protein, 306 aa, synthetic construct
  REGION 1..306: note = SARS-CoV 3CLpro
SGFRKMAFPSGKVEGCMVQVTCGTTTLNGLWLDDTVYCPRHVICTAEDMLNPNYEDLLIRKSNHSFLVQAGNVQLRVIGHSMQNCLLRLKVDTSNPKTPKYKFVRIQPGQTFSVLACYNGSPSGVYQCAMRPNHTIKGSFLNGSCGSVGFNIDYDCVSFCYMHHMELPTGVHAGTDLEGKFYGPFVDRQTAQAAGTDTTITLNVLAWLYAAVINGDRWFLNRFTTTLNDFNLVAMKYNYEPLTQDHVDILGPLSAQTGIAVLDMCAALKELLQNGMNGRTILGSTILEDEFTPFDVVRQCSGVTFQ

SEQ ID NO: 10 - protein, 97 aa, synthetic construct
  REGION 1..97: note = HIV-2 Protease
PQFSLWKRPVVTAHIEGQPVEVLLDTGADSIVAGIELGSNYSPKIVGGIGGFINTKEYKNVEIEVLNKRVRATIMTGDTPINIFGRNILASGMSLNL

SEQ ID NO: 11 - protein, 48 aa, synthetic construct
  REGION 1..48: note = CA-15
LRPGSVVVQLTLAFREGTINVHDVETQFNQYKTEAASRYNLTISDVSG

SEQ ID NO: 12 - protein, 268 aa, synthetic construct [sequence omitted]

SEQ ID NO: 13 - skipped

SEQ ID NO: 14 - protein, 7 aa, synthetic construct
  REGION 1..7: note = 3CLpro substrate fragment - enzymatic hydrolysis product carboxy terminus
KTSAVLQ

SEQ ID NO: 15 - protein, 7 aa, synthetic construct
  REGION 1..7: note = 3CLpro substrate fragment - enzymatic hydrolysis product amino terminus
SGFRKME

SEQ ID NO: 16 - protein, 11 aa, synthetic construct
  REGION 1..11: note = SARS-CoV-2 3CLpro substrate/ cleavage site nsp4/5
TSAVLQSGFRK

SEQ ID NO: 17 - protein, 11 aa, synthetic construct
  REGION 1..11: note = SARS-CoV-2 3CLpro substrate s02 in Chan et al
SGVTFQSAVKR

SEQ ID NO: 18 - protein, 11 aa, synthetic construct
  REGION 1..11: note = SARS-CoV-2 3CLpro substrate/ cleavage site nsp6/7
KVATVQSKMSD

SEQ ID NO: 19 - protein, 11 aa, synthetic construct
  REGION 1..11: note = SARS-CoV-2 3CLpro substrate/ cleavage site nsp7/8
NRATLQAIASE

SEQ ID NO: 20 - protein, 11 aa, synthetic construct
  REGION 1..11: note = SARS-CoV-2 3CLpro substrate/ cleavage site nsp8/9
SAVKLQNNELS

SEQ ID NO: 21 - protein, 11 aa, synthetic construct
  REGION 1..11: note = SARS-CoV-2 3CLpro substrate/ cleavage site nsp9/10
ATVRLQAGNAT

SEQ ID NO: 22 - protein, 11 aa, synthetic construct
  REGION 1..11: note = SARS-CoV-2 3CLpro substrate s07 in Chan et al
REPMLQSADAQ

SEQ ID NO: 23 - protein, 11 aa, synthetic construct
  REGION 1..11: note = 3CLpro cleavage site nsp12/13
PHTVLQAVGAC

SEQ ID NO: 24 - protein, 11 aa, synthetic construct
  REGION 1..11: note = 3CLpro cleavage site nsp13/14
NVATLQAENVT

SEQ ID NO: 25 - protein, 11 aa, synthetic construct
  REGION 1..11: note = 3CLpro cleavage site nsp14/15
TFTRLQSLENV

SEQ ID NO: 26 - protein, 11 aa, synthetic construct
  REGION 1..11: note = 3CLpro substrate s11 in Chan et al
FYPKLQSSQAW

SEQ ID NO: 27 - protein, 12 aa, synthetic construct
  REGION 1..12: note = 3CLpro substrate in Grum-Tokars et al.
ESATLQSGLAKS

SEQ ID NO: 28 - protein, 10 aa, synthetic construct
  REGION 1..10: note = 3CLpro substrate in Grum-Tokars et al.
LAQAVRSSSR

SEQ ID NO: 29 - protein, 12 aa, synthetic construct
  REGION 1..12: note = 3CLpro substrate in Grum-Tokars et al.
VNSTLQSGLRKM

SEQ ID NO: 30 - protein, 10 aa, synthetic construct
  REGION 1..10: note = 3CLpro substrate in Grum-Tokars et al.
SAVLQSGFRK

SEQ ID NO: 31 - protein, 12 aa, synthetic construct
  REGION 1..12: note = 3CLpro substrate in Grum-Tokars et al.
CESATLQSGLAK

SEQ ID NO: 32 - protein, 9 aa, synthetic construct
  REGION 1..9: note = 3CLpro substrate in Grum-Tokars et al.
SVTLQSGYR

SEQ ID NO: 33 - protein, 11 aa, synthetic construct
  REGION 1..11: note = SARS-CoV-2 3CLpro substrate/ cleavage site nsp5/6
SGVTFQGKFKK

SEQ ID NO: 34 - protein, 9 aa, synthetic construct
  REGION 1..9: note = 3CLpro substrate in Grum-Tokars et al.
AVLQSGFKK

SEQ ID NO: 35 - protein, 11 aa, synthetic construct
  REGION 1..11: note = SARS-CoV-2 3CLpro substrate/ cleavage site nsp10-12
REPLMQSADAS

SEQ ID NO: 36 - protein, 11 aa, synthetic construct
  REGION 1..11: note = SARS-CoV-2 3CLpro substrate/ cleavage site nsp16/15
FYPKLQASQAW

(19) United States
(12) Patent Application Publication
(10) Pub. No.: US 2024/0361320 A1
(43) Pub. Date: Oct. 31, 2024

(54) ELECTROCHEMICAL DETECTION OF A VIRAL INFECTION

(71) Applicant: Ramot at Tel-Aviv University Ltd., Tel-Aviv (IL)

(72) Inventors: Ella BORBERG, Tel-Aviv (IL); Eran GRANOT, Tel-Aviv (IL); Fernando PATOLSKY, Tel-Aviv (IL)

(73) Assignee: Ramot at Tel-Aviv University Ltd., Tel-Aviv (IL)

(21) Appl. No.: 18/768,074

(22) Filed: Jul. 10, 2024

Related U.S. Application Data
(63) Continuation of application No. PCT/IL2023/050032, filed on Jan. 10, 2023.
(60) Provisional application No. 63/297,878, filed on Jan. 10, 2022.

(51) Int. Cl. G01N 33/569; G01N 27/327; G01N 33/543

(52) U.S. Cl. CPC G01N 33/56983; G01N 27/3271; G01N 33/5438; G01N 2469/10

(57) ABSTRACT

A functionalized electrode capable of selectively interacting with a viral biomarker, an electrochemical system comprising the electrode and methods utilizing same for, for example, determining a presence of a viral infection in a subject are provided.

### RELATED APPLICATIONS

This application is a Continuation of PCT Patent Application No. PCT/IL2023/050032 having International filing date of Jan. 10, 2023, which claims the benefit of priority under 35 USC § 119(e) of U.S. Provisional Patent Application No. 63/297,878 filed on Jan. 10, 2022. The contents of the above applications are all incorporated by reference as if fully set forth herein in their entirety.

### SEQUENCE LISTING STATEMENT

The XML file, entitled 100180SequenceListing.xml, created on Jul. 10, 2024, comprising 47,528 bytes, submitted concurrently with the filing of this application is incorporated herein by reference. The sequence listing submitted herewith is identical to the sequence listing forming part of the international application.

### FIELD AND BACKGROUND OF THE INVENTION

The present invention, in some embodiments thereof, relates to electrochemical detection and, more particularly, but not exclusively, to novel system and methods for electrochemically detecting a presence of a virus, including, but not limited to, a coronavirus such as SARS-CoV-2.

SARS-CoV-2 is a coronavirus of the family Coronaviridae, and it is an enveloped positive-sense single-stranded ribonucleic acid (RNA) virus [Nat Microbiol 2020, 5, 536]. The four structural proteins are spike, envelope, membrane, and nucleocapsid. Spike protein mediates entry into host cells by binding to a cellular receptor, angiotensin-converting enzyme 2 [Verdecchia et al. European Journal of Internal Medicine 2020, 76, 14]. Then, Spike protein is cleaved by cellular cathepsin L and the transmembrane protease serine 2 [Zhou et al. Nature 2020, 579, 270]. Following the release of the viral genome into host cytosol, open reading frames in the RNA are translated into viral replicase proteins, which are cleaved into individual non-structural proteins via host and viral proteases such as 3-chymotrypsin-like protease (3CL^{pro}) [Albzeirat et al., International Journal of Multidisciplinary Sciences and Advanced Technology ISSN 2708-0587 2020, 1, Special Issue Covid-19, 1-18; and Harrison et al., Trends in Immunology 2020, 41, 1100], forming the RNA polymerase [Perlman and Netland, Nat Rev Microbiol 2009, 7, 439]. Replicase components additionally cause a change in the endoplasmic reticulum forming double-layered vesicles, facilitating viral genomic replication and virion formation [Harrison et al. 2020 supra; and Snijder et al., Journal of Virology 2006, 80, 5927].

COVID-19 (Coronavirus disease 2019), the disease caused by severe acute respiratory syndrome coronavirus 2 (SARS-CoV-2), was acknowledged by the World Health Organization (WHO) as a pandemic outbreak on March 2020, causing over 4.4 million deaths as of August 2021, with worldwide health and economic effects that are expected to persist for years to come [Kissler, et al, Science 2020, 368, 860].

In hopes of ending the pandemic, attempts to lower transmission rates have been implemented. Unfortunately, SARS-CoV-2 transmission restriction by traditional countermeasures, based on isolating symptomatic individuals, is ineffective since a large percentage of infections is caused by asymptomatic carriers, thus counteractions taken to curb the COVID-19 pandemic depend on strict strategies for high-quality testing procedures. These procedures mainly target specific viral molecules for identifying infected carriers.

The primary detection methods currently include reverse transcription-polymerase chain reaction (RT-PCR), a well-established sensitive diagnostic method nearly reaching single-molecule sensitivity [Corman et al., Euro Surveill. 2020, 25, 3, 2000045], and uses non-invasive sampling such as saliva, or throat and nasal swabs. However, RT-PCR requires high-end equipment, not suitable for point-of-care (POC) setting, and intricate sample processing by specialized lab personnel, thus representing a time-consuming approach. The required logistics delays screening results even further, typically given after several hours or even days after sampling, hindering the timeliness of reactive measures, thus allowing for larger transmission chains [Ferretti et al., Science. 2020, 368, 6491, eabb6936]. Additionally, PCR is susceptible to foreign nucleic acid contamination, non-specific amplification [Orooji et al. Nano-Micro Lett. 2020, 13, 18], and the presence of viral genomic material alone does not indicate active infection, possibly marking non-infectious individuals [Wu et al., BMC Medicine 2021, 19, 77; and Alexandersen et al., Nat Commun 2020, 11, 6059].

Immunoassay approaches like enzyme-linked immunoassays (ELISA), which work based on antigen-antibody interactions, are highly sensitive and much quicker than PCR. However, immunoassays require specific and high-affinity antibodies (and sometimes expensive recombinant and conjugated antibodies), especially in the case of complex investigations, which has limited their application in routine point-of-care procedures. For solving this problem, low-cost analogs of antibodies have gained much attention in experimental studies. Multiple antibody tests have been developed to detect SARS-CoV-2, including lateral flow immunoassay (LFIA), chemiluminescence enzyme immunoassay (CLIA), and fluorescence enzyme-linked immunoassay (FIA). The majority of these assays use spike or nucleocapsid proteins of SARS-CoV-2 [Amanat et al., Nat Med 2020, 26, 1033; and Bryant et al. Sci Immunol. 2020, 5, 47, eabc6347] to detect immunoglobulin G (IgG) and/or immunoglobulin M (IgM) antibodies produced by the host immune system against the virus. The reported methods are relatively fast (several minutes), and many are compatible with POC approaches. However, the most applicable test for POC approaches, LFIA, reportedly has the lowest performance [Vengesai et al., Systematic Reviews 2021, 10, 155], and these quantitative and qualitative assays detect exposure to SARS-CoV-2 by antibody responses rather than active infection. This can aid in the identification of factors that correlate with effective immunity to SARS-CoV-2 [Martinez-Fleta et al., SARS-Cov-2 Cysteine-like Protease (Mpro) Is Immunogenic and Can Be Detected in Serum and Saliva of COVID-19-Seropositive Individuals, Infectious Diseases (Except HIV/AIDS), 2020], but then again is less suitable for diagnosing infectious individuals [P. O. of the E. Union, “C/2020/2391, Communication from the Commission Guidelines on COVID-19 in vitro diagnostic tests and their performance 2020/C 122 I/01”, Official Journal of the European Union, 2020, C-122-I, 1-7].

3CL^{pro} is a viral proteolytic enzyme that belongs to the cysteine protease class [Jin et al., Nature 2020, 582, 289; and Rawlings et al., Nucleic Acids Research 2014, 42, D503], and acts as a catalyst for peptide bond hydrolysis of viral polyproteins.

Since 3CL^{pro} is a non-structural protein, it is not exposed in the viral particle; therefore, it is not prone to linger in host fluids as do viral envelope fragments. Moreover, since 3CL^{pro} carries out a critical function in viral replication, its activity is essential for the viral life cycle; thus, its presence is indicative of an active infection [Harrison et al. 2020 supra]. As a critical part of viral proliferation, meaning active infection, 3CL^{pro} has been extensively studied in coronaviruses, past and current, as a target for treatment [Zhu et al., ACS Pharmacol. Transl. Sci. 2020; Morse et al. Chembiochem. 2020, 21, 5, 730-738.; and Zhang et al., Science 2020, 368, 409].

SARS-CoV-2 proteins are expressed as a single polypeptide chain that is cleaved in eleven specific sites [Ghosh et al. Biochim Biophys Acta Biomembr. 2018, 1860, 2, 335-346]. 3CL^{pro} cleaves at specific sites of amino acid sequences, usually in the LQ*S pattern, S could be replaced with either A or G (cleaving site is marked with *) [Zhang et al., 2020, supra; and Senger, et al., Mem. Inst. Oswaldo Cruz 2020, 115]. The 3CL^{pro} catalytic site holds a catalytic dyad of C—H. The hydrolysis is catalyzed in a well-known nucleophilic reaction. First, C thiol is deprotonated by H residue, causing a nucleophilic attack of the substrates carbonyl carbon by the anionic sulfur, followed by the N-terminus of the substrate being protonated by the H residue of the catalytic site and detaching from the substrate. The C-terminus of the substrate forms thioester intermediate with C residue, which is then hydrolyzed to produce a carboxylic acid and regenerate the catalytic site. The carboxylic acid product may cause an in-vitro pH drop in a non-buffered medium [Wang et al., ACS Catal. 2020, 10, 5871; and Huang et al., Biochemistry 2004, 43, 4568].

Proteases have been recognized as essential biomarkers in many conditions, including cancer [Edwards and Murphy, Nature 1998, 394, 527], Alzheimer's [Cataldo and Nixon, PNAS 1990, 87, 3861], AIDS [Andrew et al., Current Topics in Medicinal Chemistry 2005, 5, 1589], and inflammation [Funovics et al., Anal Bioanal Chem 2003, 377, 956], and hence studies aimed targeting proteases as a target of drugs and as a diagnostic tool have been extensively conducted [B. Turk, Nat Rev Drug Discov 2006, 5, 785].

Protease detection assays could be grouped into affinity and activity assays. Since affinity assays detect protease regardless of activity, activity assays are more applicable for functional protease detection. Activity assays include colorimetric [Zhou, et al., Analyst 2014, 139, 1178], mass spectrometry-based [Hu et al., Anal. Chem. 2015, 87, 4409], and fluorescence resonance energy transfer assays [Liu et al., Biochemical and Biophysical Research Communications 2005, 333, 194]. These can achieve low detection limits (at the pM range) but cannot be applied in multiplexed sensing platforms since only a few probes can generate different signals.

More recently, nanomaterials such as noble metal nanoparticles [Kim et al., Anal. Chem. 2014, 86, 3825], quantum dots [Wu et al., Anal. Chem. 2014, 86, 10078], and graphene oxide [Jin et al., ACS Nano 2012, 6, 4864] have been introduced in protease assays with impressive detection limits and more multiplexing capabilities. However, these are prone to limitations in the stability of the reporter molecules.

An additional group of assays, in which the substrate is immobilized on the array's surface, includes electrochemical [Cao et al., Biosensors and Bioelectronics 2013, 45, 1], surface-enhanced Raman scattering [Chen et al., Nanoscale 2013, 5, 5905], and surface plasmon resonance assays [Tripathi et al, International Journal of Biological Macromolecules 2020, 164, 2622]. These provide a platform for proteases detection that could be easily multiplexed. Nonetheless, the sensitivity of these assays tends to be lower due to the substrate immobilization onto the detection surface, causing only proteases near surfaces to elicit a signal.

Yakoh et al. [Biosensors and Bioelectronics 2021, 176, 15, 112912] describes electrochemical detection of SARS-CoV-2 antibodies or spike protein using a device comprising graphene oxide (GO)-embedded cellulose paper electrodes immobilized with SARS-CoV-2 spike protein-containing receptor-binding domain (SP RBD), and blocked with skim milk (SKI). The presence of SARS-CoV-2 in the sample therein results in the formation of a rigid antigen-antibody complex that reduces the charge transfer of the redox probe, and the electrochemical response is monitored using square-wave voltammetry (SMW) technique.

### SUMMARY OF THE INVENTION

The present inventors have devised and successfully practiced an ultra-fast electrochemical approach targeting a viral biomarker such as, for example, a SARS-CoV-2-specific proteolytic enzyme, 3CL^{pro}, for detecting an active infection in a subject. Both the presence and activity of the viral biomarker (e.g., 3CL^{pro}) in saliva are detected by a change in the cyclic voltammetry (CV) signal of an agent such as p-benzoquinone, that performs as a reduction-oxidation (RedOx) in response to a presence of the viral biomarker (e.g., pH change).. The present inventors have utilized carbon paper electrodes (CPE), preferably featuring a very high surface area, combined with the intrinsic CV fast detection turnover, sensitivity, selectivity, and enzymatic signal amplification, to provide fast and effective detection of a viral infection, for example, within 1 minute, directly from unprocessed biological samples, such as saliva swab samples.

According to an aspect of some embodiments of the present invention there is provided an electrode (e.g., a carbon electrode) having attached (e.g., physically attached, for example, adsorbed or otherwise associated with) thereto an agent that specifically binds to a biomarker of a SARS-CoV-2 viral infection, wherein the biomarker is found in a saliva of a subject having an active SARS-CoV-2 viral infection.

According to some of any of the embodiments described herein, the biomarker is a SARS-CoV-2-specific proteolytic enzyme proteolytic enzyme According to some of any of the embodiments described herein, the SARS-CoV-2-specific proteolytic enzyme is 3CL^{pro} (SARS-CoV-2 3CL^{pro}).

According to some of any of the embodiments described herein, the 3CL^{pro} comprises an amino acid sequence as set forth in SEQ ID NO. 2.

According to some of any of the embodiments described herein, the agent that specifically binds to the biomarker is an antibody specific to the biomarker.

According to some of any of the embodiments described herein, the agent that specifically binds to the biomarker is an antibody specific to the proteolytic enzyme.

According to some of any of the embodiments described herein, the agent that specifically binds to the biomarker is an antibody specific to the SARS-CoV-2 3CL^{pro}.

According to some of any of the embodiments described herein, the antibody binds to a portion of the amino acid sequence as set forth in SEQ ID NO: 2, the portion having an amino acid sequence as set forth in SEQ ID NO: 3.

According to an aspect of some embodiments of the present invention there is provided an electrode (e.g., a carbon electrode) having attached (e.g., physically attached, for example, adsorbed or otherwise associated with) thereto an agent that specifically binds to a biomarker of a viral infection, wherein: the biomarker is a proteolytic enzyme indicative of the viral infection; and/or the biomarker is found in a saliva of a subject having the viral infection.

According to some of any of the embodiments described herein, the biomarker is the proteolytic enzyme.

According to some of any of the embodiments described herein, the agent that specifically binds to the biomarker is an antibody specific to the proteolytic enzyme.

According to some of any of the embodiments described herein, the biomarker is selected from an enzyme, an antigen, an antibody, and a biomarker of viral replication.

According to some of any of the embodiments described herein, the biomarker is a SARS-CoV-2-specific proteolytic enzyme.

According to some of any of the embodiments described herein, the agent that specifically binds to the proteolytic enzyme is an antibody specific to the SARS-CoV-2-specific proteolytic enzyme.

According to some of any of the embodiments described herein, the SARS-CoV-2-specific proteolytic enzyme is 3CL^{pro} (SARS-CoV-2 3CL^{pro}).

According to some of any of the embodiments described herein, the 3CL^{pro} comprises an amino acid sequence as set forth in SEQ ID NO: 2.

According to some of any of the embodiments described herein, the agent that specifically binds to the biomarker is an antibody specific to the SARS-CoV-2 3CL^{pro}.

According to some of any of the embodiments described herein, the antibody binds to a portion of the amino acid sequence as set forth in SEQ ID NO: 2, the portion having an amino acid sequence as set forth in SEQ ID NO: 3.

According to some of any of the embodiments described herein, the electrode is a carbon electrode and in some embodiments, the carbon electrode is a carbon paper electrode.

According to some of any of the embodiments described herein, the carbon electrode is a carbon fiber microelectrode.

According to some of any of the embodiments described herein, the electrode is further having attached (e.g., physically) thereto an agent that inhibits attachment (e.g., physical, adsorption) of proteins other than the biomarker to the electrode.

According to an aspect of some embodiments of the present invention there is provided an electrochemical system comprising the electrode (e.g., carbon electrode) as described herein in any of the respective embodiments and any combination thereof.

According to some of any of the embodiments described herein, the electrochemical system is configured such that when the viral biomarker is contacted with the electrode, a detectable change in an electrochemical parameter is generated.

According to some of any of the embodiments described herein, the electrode forms a part of an electrochemical cell and the electrochemical cell is operable by electrically connecting the electrode to a power source.

According to some of any of the embodiments described herein, the electrochemical cell further comprises a reference electrode and optionally an auxiliary electrode.

According to some of any of the embodiments described herein, the electrochemical cell is operable by contacting the electrode with an electrolyte.

According to some of any of the embodiments described herein, the electrochemical system further comprises the electrolyte.

According to some of any of the embodiments described herein, the electrolyte comprises a substance that is capable of interacting (e.g., selectively) with the biomarker, wherein a detectable change is an electrochemical parameter is generated in response to an interaction between the biomarker and the substance.

According to some of any of the embodiments described herein, the biomarker is a proteolytic enzyme and the substance is a substrate of the proteolytic enzyme.

According to some of any of the embodiments described herein, the electrolyte further comprises an electroactive agent that undergoes an electrochemically detectable (e.g., redox) reaction in response to the interaction, to thereby generate the change in the electrochemical parameter.

According to some of any of the embodiments described herein, the biomarker, the substance and the electroactive agent are selected such that the interaction between the biomarker and the substance generates a moiety or species, and the electroactive agent undergoes an electrochemically detectable (e.g., redox) reaction in response to a presence of the chemical moiety or species.

According to some of any of the embodiments described herein, the chemical moiety or species comprises a proton.

According to some of any of the embodiments described herein, the interaction between the biomarker and the substance results in a pH change and wherein the electroactive agent undergoes a pH-dependent electrochemically detectable (e.g., redox) reaction.

According to an aspect of some embodiments of the present invention there is provided a method of determining a presence and/or amount of a viral biomarker in a sample, the method comprising contacting the sample with the electrode as described herein in any of the respective embodiments, and determining a change in an electrochemical parameter generated upon operating an electrochemical system as described herein in any of the respective embodiments, wherein the change is indicative of the presence and/or amount of the viral biomarker in the sample.

According to some of any of the embodiments described herein, the sample is a biological sample drawn from a subject, the method being for determining a presence and/or amount of a viral infection in the subject.

According to some of any of the embodiments described herein, the biological sample is a saliva sample of the subject.

According to some of any of the embodiments described herein, a pH of the saliva of the subject is in a range of from 6 to 8.

According to some of any of the embodiments described herein, the biomarker is SARS-CoC-2 3CL^{pro}, the method being of determining a presence and/or amount of a viral infection caused by SARS-CoV-2 in the subject.

According to an aspect of some embodiments of the present invention there is provided a method of determining a presence of a viral infection associated with 3CL^{pro} in a subject, the method comprising contacting a saliva sample of the subject with a probe selective to the 3CL^{pro}, the probe being such that generates a detectable signal in response to a presence of 3CL^{pro} in the sample.

According to an aspect of some embodiments of the present invention there is provided a carbon electrode (e.g., a carbon paper electrode, preferably featuring high surface area) having attached thereto an agent that specifically binds to a viral biomarker, as described herein, also referred to herein as an immune-functionalized carbon electrode.

According to some of any of the embodiments described herein, the viral biomarker is a proteolytic enzyme.

According to some of any of the embodiments described herein, the agent that specifically binds to the viral biomarker is an antibody specific to the enzyme.

According to some of any of the embodiments described herein, the viral biomarker is a SARS-CoV-2-specific proteolytic enzyme, e.g., 3CL^{pro}.

According to some of any of the embodiments described herein, the agent that specifically binds to the proteolytic enzyme is an antibody specific to the SARS-CoV-2-specific proteolytic enzyme, e.g., 3CL^{pro}.

According to an aspect of some embodiments of the present invention there is provided an electrochemical system comprising a carbon electrode as described herein According to some of any of the embodiments described herein, the system further comprises an electrolyte.

According to some of any of the embodiments described herein, the system further comprises an electroactive agent that undergoes a redox reaction in response to an interaction between the viral biomarker and the agent that specifically binds it.

According to some of any of the embodiments described herein, the biomarker and the agent that specifically binds thereto are selected such that an interaction therebetween generates a chemical species.

According to some of any of the embodiments described herein, the electroactive agent undergoes a redox reaction in the presence of the chemical species.

According to some of any of the embodiments described herein, the chemical species comprises protons.

According to some of any of the embodiments described herein, the interaction results in a pH change and wherein the electroactive agent undergoes a pH-dependent redox reaction.

According to an aspect of some embodiments of the present invention there is provided a method of determining a presence of a viral infection in a subject, the method comprising contacting a biological sample that comprises the viral biomarker (e.g., a saliva sample) of the subject with the electrode as described herein.

According to some of any of the embodiments described herein, the method further comprises assembling the electrode in an electrochemical system as described herein, and determining a change in electrochemical parameter.

According to an aspect of some embodiments of the present invention there is provided a method of determining a presence of a viral infection caused by SARS-CoV-2 in a subject, the method comprising determining a presence of 3CL^{pro} as described herein in a saliva sample of the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D describe the CPE surface, immuno-functionalization, and biosensor method. FIG. 1A is a photograph of an exemplary CPE. Blue inset: SEM images of the detection window, scale bar: 1 mm. Green inset: SEM images of 3D microfiber matrix of CPE, scale bar: 50 μm. FIG. 1B presents a schematic illustration of an exemplary CPE immuno-functionalization according to some embodiments of the present invention. FIG. 1C is a photograph illustrating saliva sampling by oral cavity swabbing with a CPE according to some of the present embodiments. FIG. 1D is a schematic illustration of a biosensor SARS-CoV-2 detection method according to some embodiments of the present invention.

FIGS. 2A-F present the characterization of para-benzoquinone (pBQ) as an exemplary RedOx pH indicator. FIG. 2A is a bar graph showing the measured pH change caused by 3CL^{pro} (1 μM) activity in the presence of 3CL^{pro} substrate (100 μM, orange plot) and the absence of 3CL^{pro} substrate (green plot). FIG. 2B presents a pH-dependent pBQ RedOx reaction. FIG. 2C presents CV curves of pBQ (15 μM) in PB (900 μl of 25 mM) and NaCl (75 mM), at pH values varying between 5.35 and 8.10. Scan rate: 0.1 V sec⁻¹, vs. Ag/AgCl, using untreated CPE as the working electrode. FIG. 2D presents linear plots showing shifts in the potential of CV peaks of oxidation (black) and reduction (red) based on values measured at pH 8.10 as described for FIG. 2C. FIG. 2E presents a calibration curve of 8-Hydroxypyrene-1,3,6-trisulfonic acid (HPTS) fluorescence as a function of pH, which was used to measure pH change. FIG. 2F presents CV of untreated CPE obtained with 3CL^{pro} (black curve) or with 3CL^{pro} and its substrate, 3CL^{pro}-substrate (SEQ ID NO: 1) (red curve), in the absence of pBQ. Untreated CPE used as the working electrode, 900 μl of 80 nM 3CL^{pro}, 25 mm PB, 75 mM NaCl, pH 7.8, scan rate 0.1 V sec⁻¹, vs. Ag/AgCl.

FIGS. 3A-H present the performance of immuno-functionalized CPE biosensors. FIG. 3A presents an adsorption curve of 3CL^{pro}-specific IgG antibody (binding to SEQ ID NO: 3) onto clean CPE over time. FIG. 3B presents a non-specific protein binding curve of CA-15.3 (SEQ ID NO: 11) onto CPE treated with the 3CL^{pro}-specific IgG (binding to SEQ ID NO: 3) with (black curve) and without (red curve) BSA blocking. FIG. 3C presents a specific protein binding curve of 3CL^{pro} onto CPE treated with 3CL^{pro}-specific IgG (binding to SEQ ID NO: 3) and BSA (SEQ ID NO: 4). Inset: enlarged view of the dotted area. FIG. 3D presents CV curves of CPE treated with 3CL^{pro} antibody (binding to SEQ ID NO: 3) and exposed to SARS-CoV-2 negative saliva before (black) and after (red) exposure to 3CL^{pro} substrate (SEQ ID NO: 1). FIG. 3E presents CV curves of CPE treated with 3CL^{pro}-specific antibody (binding to SEQ ID NO: 3) and exposed to SARS-CoV-2 negative saliva spiked with 0.2 pmol 3CL^{pro} (SEQ ID NO: 2) before (black) and after (red) exposure to 3CL^{pro} substrate (SEQ ID NO: 1). FIG. 3F presents CV curves of CPE treated with 3CL^{pro}-specific antibody (binding to SEQ ID NO: 3) and exposed to PCR SARS-CoV-2 positive saliva, before (black) and after (red) exposure to 3CL^{pro} substrate (SEQ ID NO: 1). FIG. 3G presents CV curves of CPE treated with myoglobin antibody (targeting SEQ ID NO: 5) and exposed to SARS-CoV-2 negative saliva spiked with 0.2 pmol 3CL^{pro} (SEQ ID NO: 2) before (black) and after (red) exposure to 3CL^{pro} substrate (SEQ ID NO: 1), showing antibody specificity. FIG. 3H presents CV curves measured for CPE immuno-functionalization steps; Untreated (black); treated with 3CL^{pro}-specific antibody (binding to SEQ ID NO: 3) (red); and treated with 3CL^{pro}-specific antibody and then with BSA (SEQ ID NO: 4) (blue). All CV curves were obtained in 900 μl of 15 μM pBQ, 25 mM PB, 75 mM NaCl, pH 7.4, scan rate 0.1 V sec⁻¹, vs. Ag/AgCl.

FIGS. 4A-C present SARS-CoV-2 detection in clinical samples. FIG. 4A is a bar graph showing pBQ oxidation peak shift of healthy (blue) and PCR SARS-CoV-2 positive (red) saliva samples. FIG. 4B presents a scatter plot of pBQ oxidation peak shift of SARS-CoV-2 negative saliva (blue, N=24), the saliva of recovered COVID-19 patients (green, N=4), SARS-CoV-2 negative saliva spiked with 3CL^{pro} (SEQ ID NO: 2) (orange, N=7), and PCR SARS-CoV-2 positive (red, N=26) saliva samples. Horizontal lines represent mean peak shift values. FIG. 4C presents pBQ oxidation peak shift results of 10 consecutive experiments measuring the same healthy saliva sample, compared with the mean value of measurements of PCR SARS-CoV-2 positive saliva samples (on the right).

FIG. 5 presents CV curves of different cycles of measuring pBQ (15 μM) in PB (900 μl, 25 mM) and NaCl (75 mM), pH 7.65. Scan rate: 0.1 V sec⁻¹, vs. Ag/AgCl, demonstrating the measurement coherence.

FIGS. 6A-J present the characterization of immuno-functionalized CPEs. FIGS. 6A-B are fluorescence microscopy images of untreated (bare) CPE (FIG. 6A) and GFP (SEQ ID NO: 6)-modified CPE (FIG. 6B) measured in PBS, scale bar: 1 mm. FIG. 6C is a comparative GFP fluorescence intensity curve of untreated (bare; red plot) and GFP (SEQ ID NO: 6)-modified (black plot) CPE, showing protein permeability through CPE; data correspond to FIGS. 6A-B. FIG. 6D is a desorption curve of the 3CL^{pro}-specific antibody (binding to SEQ ID NO: 3) from CPE over time. Desorption following soaking of the CPE in protein solution was calculated using equation (1), as described in the Method section. FIGS. 6E-F are HR-SEM images of surfaces of untreated (bare) CPE (FIG. 6E) and CPE treated with 3CL^{pro} antibody (binding to SEQ ID NO: 3) (FIG. 6F), scale bar: 100 nm. FIGS. 6G-H are representative X-Ray Photoelectron Spectroscopy (EDS) spectra for untreated (bare) (FIG. 6G) and immuno-functionalized (FIG. 6H) CPE. FIGS. 61-J are representative energy-dispersive X-ray spectroscopy (XPS) spectra of untreated CPE (bare) (FIG. 6I) and immuno-functionalized CPE (FIG. 6J).

FIG. 7A presents CV curves of exemplary immuno-functionalization steps according to the present embodiments, showing data obtained for untreated (bare) CPE (black), CPE functionalized with 3CL^{pro}-specific antibody (binding to SEQ ID NO: 3) (red), immuno-functionalized CPE blocked with BSA (SEQ ID NO: 4) (green), and immuno-functionalized and blocked CPE 2-minutes after exposure to 3CL^{pro} (SEQ ID NO: 2) (blue). CV curves were obtained in 900 μl of 10 mM [Fe(CN)₆]³⁻/[Fe(CN)₆]⁴⁻ (1:1), 0.1 M PB, 0.1 M NaCl, pH 7.0, scan rate 0.1 V sec⁻¹, vs. Ag/AgCl.

FIG. 7B presents concentration-dependent curves showing a specific protein-binding of 1-500 μg ml⁻¹ 3CL^{pro} (SEQ ID NO: 2) onto CPE treated with 3CL^{pro}-specific IgG (binding to SEQ ID NO: 3) and BSA (SEQ ID NO: 4).

FIG. 8A is a scatter plot showing pBQ oxidation peak shift of healthy saliva spiked with SARS-CoV-2 3CL^{pro} (SEQ ID NO: 2) 50 μg ml⁻¹, measured at different times from saliva spiking. After spiking, the spiked saliva sample was stored at 4° C. Data points represent mean±SD from three technical repetitions.

FIG. 8B is a scatter plot showing pBQ oxidation peak shift of healthy saliva spiked with SARS-CoV-2 3CL^{pro} (SEQ ID NO: 2) 50 μg ml⁻¹ measured at different times from CPE immuno-functionalization. After immuno-functionalization, CPEs were stored at 4° C. Data points represent mean±SD from three technical repetitions.

FIG. 8C is a scatter plot showing pBQ oxidation peak shift of healthy saliva (blue curve), and of healthy saliva spiked with 3CL^{pro} (SEQ ID NO: 2) 80 μg ml⁻¹ (black curve) from different individuals with different initial salivary pH. Data points represent mean±SD from three technical repetitions.

FIGS. 9A-C present SARS-CoV-2 detection in clinical samples. FIG. 9A is a scatter plot presenting peak shift as a function of 3CL^{pro} (SEQ ID NO: 2) concentration. FIG. 9B is a scatter plot presenting peak shift over time from infection of one individual compared with PCR and antigen test results. FIG. 9C are photographs of COVID-19 Antigen Rapid Test results in different days following infection (indicated in each inset) of the individual subject, as described in FIG. 9B.

FIG. 10 is a bar graph showing the oxidation peak shift from healthy saliva spiked with different proteases: human immunodeficiency virus (HIV) protease (SEQ ID NO: 10), the human proteases chymotrypsin (SEQ ID NO: 12) and TMPRSS2 (SEQ ID NO: 7), and 3CL^{pro}from SARS-CoV-2, SARS-CoV and MERS (SEQ ID NOs: 2, 9 and 8, respectively). Columns represent mean±SD from three distinct biological replicates.

FIG. 11 is a simplified schematic presentation of an exemplary electrochemical cell according to some of the present embodiments.

FIG. 12 is a simplified flow chart presenting an exemplary method according to some of the present embodiments.

### DESCRIPTION OF SPECIFIC EMBODIMENTS OF THE INVENTION

The present invention, in some embodiments thereof, relates to electrochemical detection and, more particularly, but not exclusively, to novel system and methods for electrochemically detecting a presence of a virus, including, but not limited to, a coronavirus such as SARS-CoV-2.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Although highly accurate virus (e.g., SARS-CoV-2) detection is achieved by methods such as RT-PCR, these are unsuitable for point-of-care (POC) applications, due to exceedingly low detection turnover rate and the requirements of expensive machinery, trained personnel, and multiple expensive and sensitive reagents.

The present inventors have devised and successfully practiced an ultra-fast electrochemical approach targeting a SARS-CoV-2-specific proteolytic enzyme, 3CL^{pro} (also referred to herein as 3CL protease) as a marker of active infection. This represents the first demonstration on the presence of this biomarker in saliva samples of infected individuals, as well as its application as a specific biomarker for the early detection of SARS-CoV-2. The self-amplifying proteolytic activity of 3CL^{pro} is detected directly from untreated saliva samples using a 3D conductive paper matrix preferably featuring high surface area, and a redox pH-indicator, within less than one minute of sample incubation. The 3D conductive paper serves both as an ultra-fast capturing surface, allowing the seconds-long rapid capturing of the biomarker molecules, and as the sensing agent, with no sample manipulation steps required. The 3CL^{pro} captured proteolytic molecules serve as self-amplification agents, thus making this platform a label-free approach for viral detection. Notably, the 3D conductive matrix is used both as sample collection and direct detection element, and due to its morphological attributes allows for the fastest detection turnover rate reported by another common approaches, with a full cycle of detection practically performed within less than one minute.

The present inventors have successfully proved the potential of the immuno-functionalized 3D conductive electrodes as a platform for the reliable and ultrafast detection of SARS-CoV-2 directly from saliva swab samples within less than one minute, using a single antibody agent. Preliminary measurements of SARS-CoV-2 positive and healthy saliva samples established the methods' accuracy and sensitivity, equivalent to laboratory RT-PCR. The detection based on 3CL^{pro} activity could potentially be more reliable, as detecting RNA may give false-positive results by detecting viral RNA fragments residues also after the infection is no longer active [Alexandersen et al., 2020, supra].

The presence and activity of 3CL^{pro} in saliva samples of SARS-CoV-2 positive individuals, which could be further detected by a change in the CV characteristics of the RedOx label pBQ, has been demonstrated. This is the first demonstration of the presence of 3CL^{pro} in salivary samples from SARS-CoV-2 patients.

All measurements were taken after one minute of exposure to saliva samples. This detection time could be further shortened by modulating the surface area, antibody density, and cell volume of the detection set-up to potentially reach detection turnover cycles of several tens of seconds.

Combined with low device costs and easily scalable multiplexed equipment, the herein disclosed methodology provides a large-scale, fast, and accurate SARS-CoV-2 detection platform, thus allowing timely implementation of measures to curb pandemic progression.

The present inventors have designed a modified carbon electrode that specifically binds a viral biomarker and exhibits a detectable change in an electrochemical parameter in the presence of the viral biomarker.

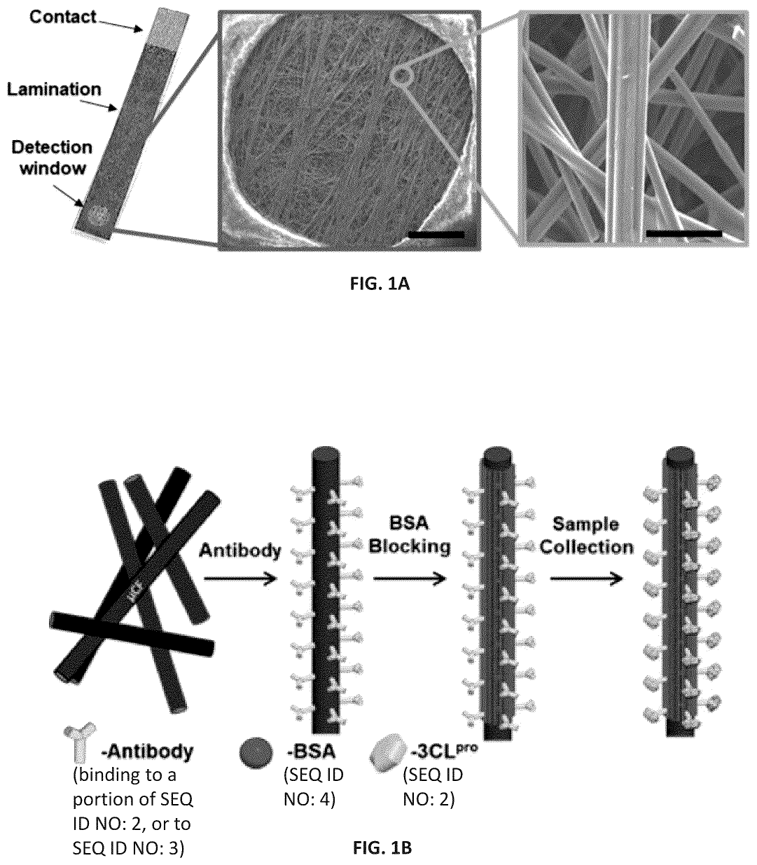

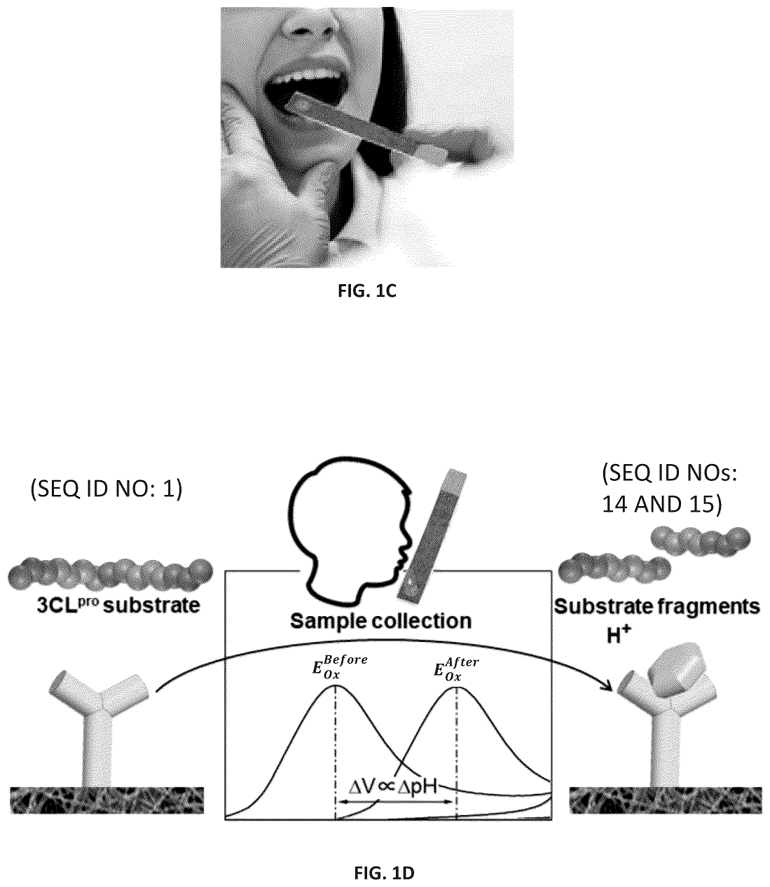

An exemplary carbon electrode is a carbon paper electrode, CPE, as presented in FIG. 1A. The modifications of the carbon electrode include (a) immuno-functionalization, for the specific binding of the viral biomarker; and optionally (b) blocking of the open binding sites in the CPE (with, e.g., BSA; SEQ ID NO: 4). A specific (selective) attachment of a viral biomarker (e.g., SARS-CoV-2 3CL^{pro}; SEQ ID NO: 2) is then possible, as illustrated in FIG. 1B. Sample collection and viral biomarker detection using the exemplary immuno-functionalized carbon electrode are illustrated in FIGS. 1C-D and generally described in Example 1.

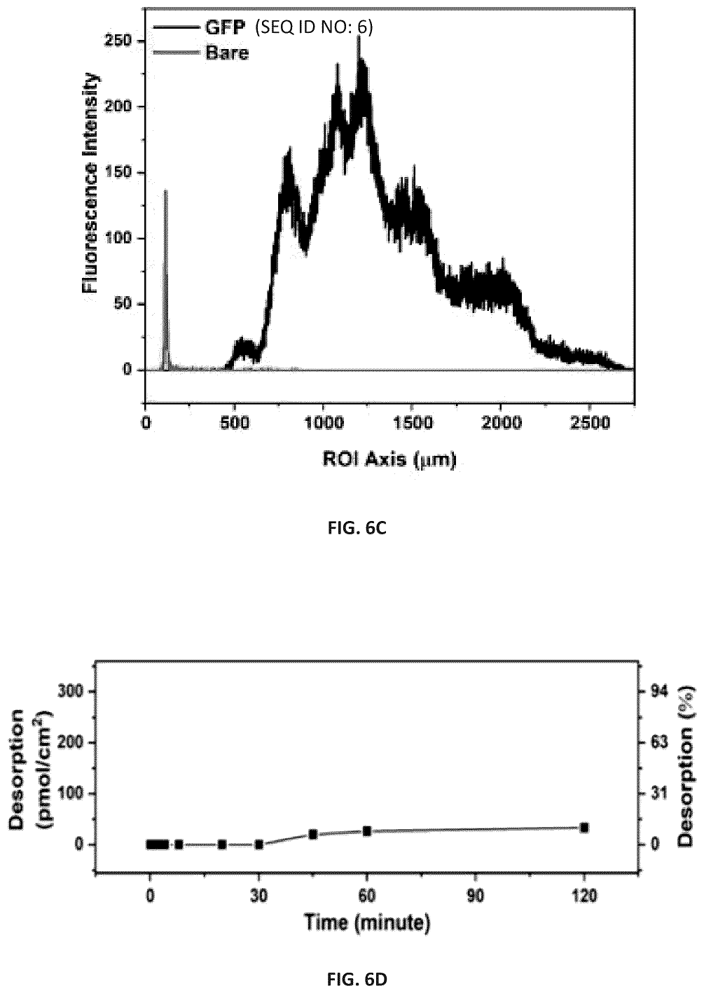

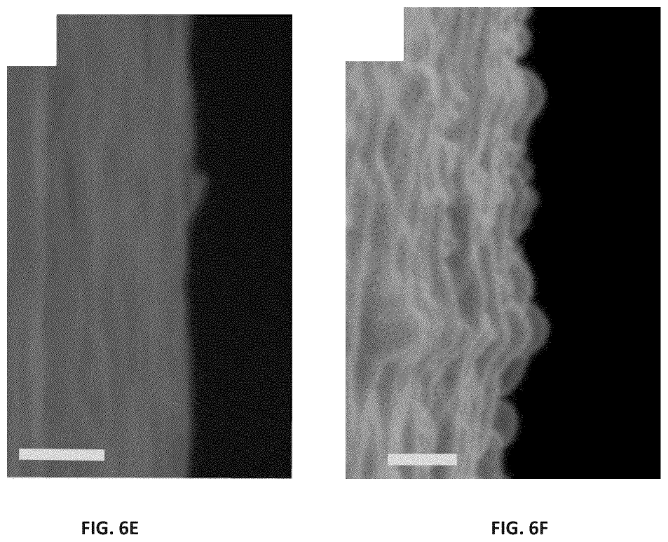

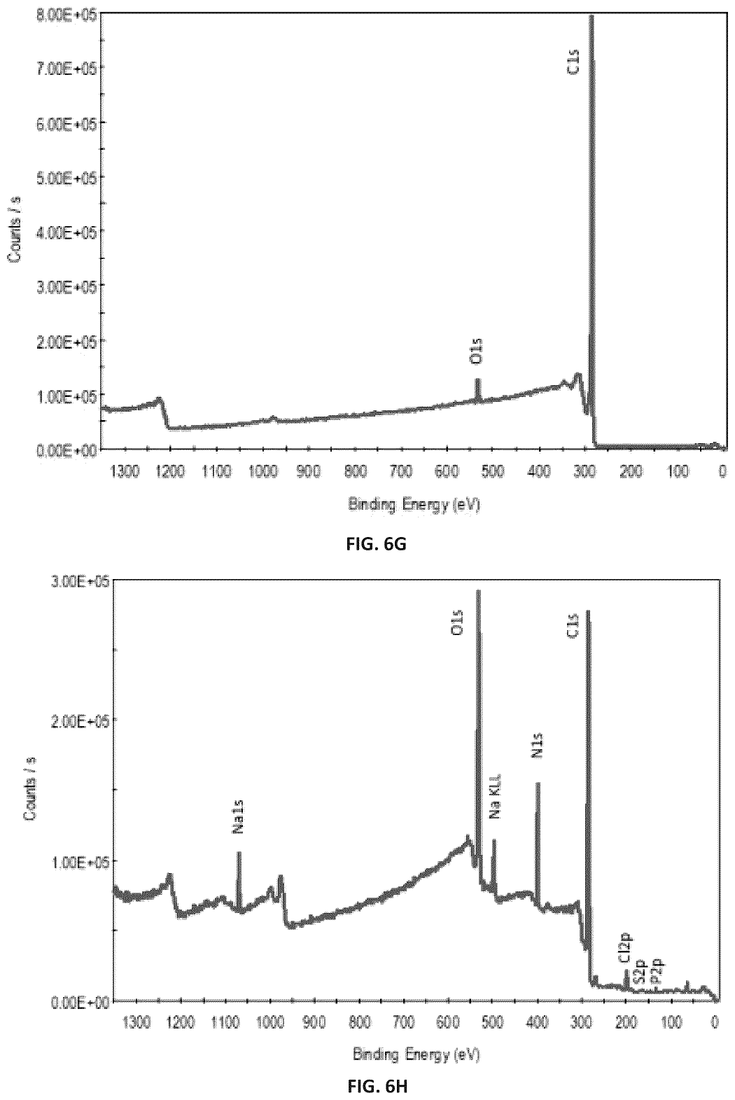

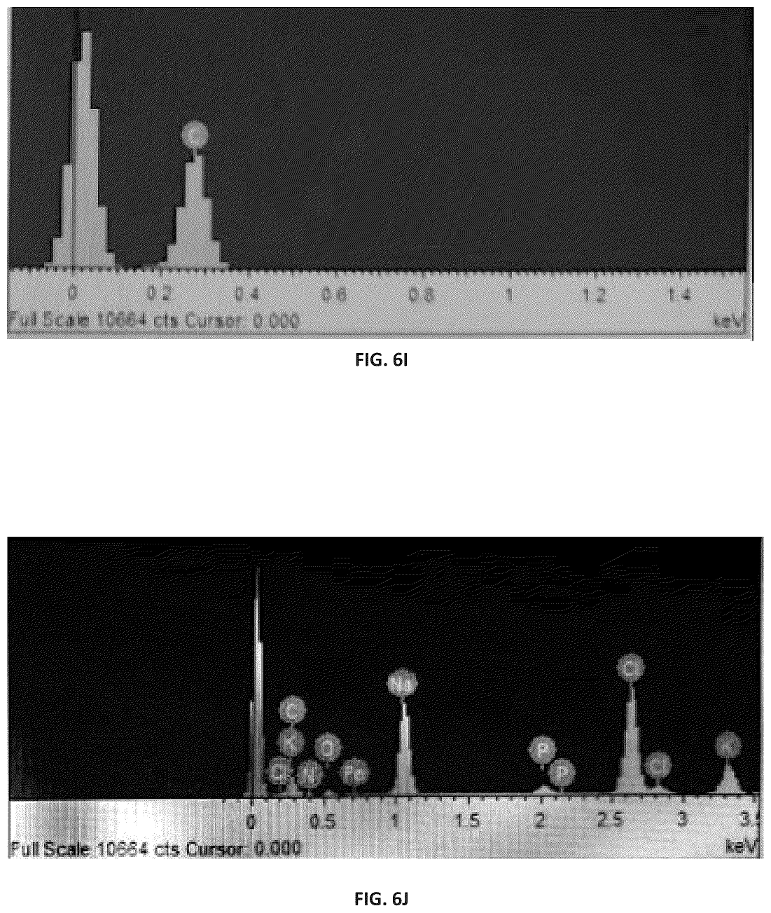

A further characterization of the exemplary immune-functionalized electrode is presented in FIG. 6A-J.

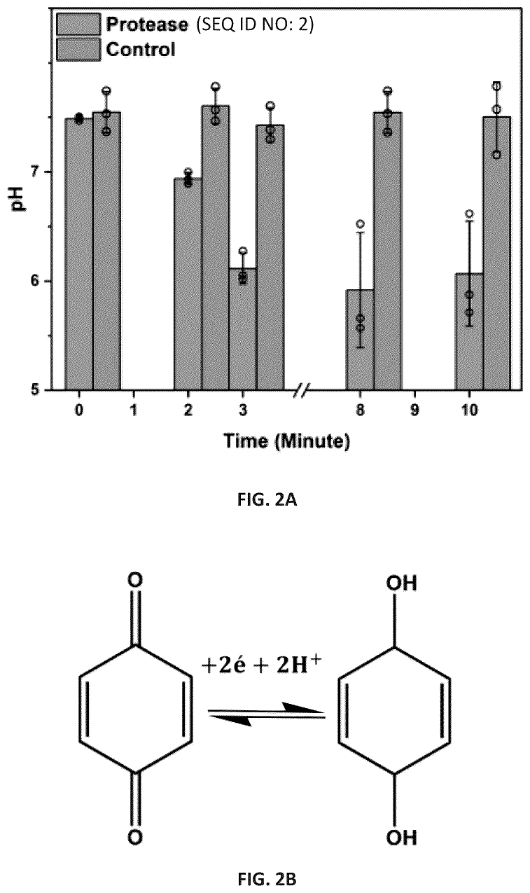

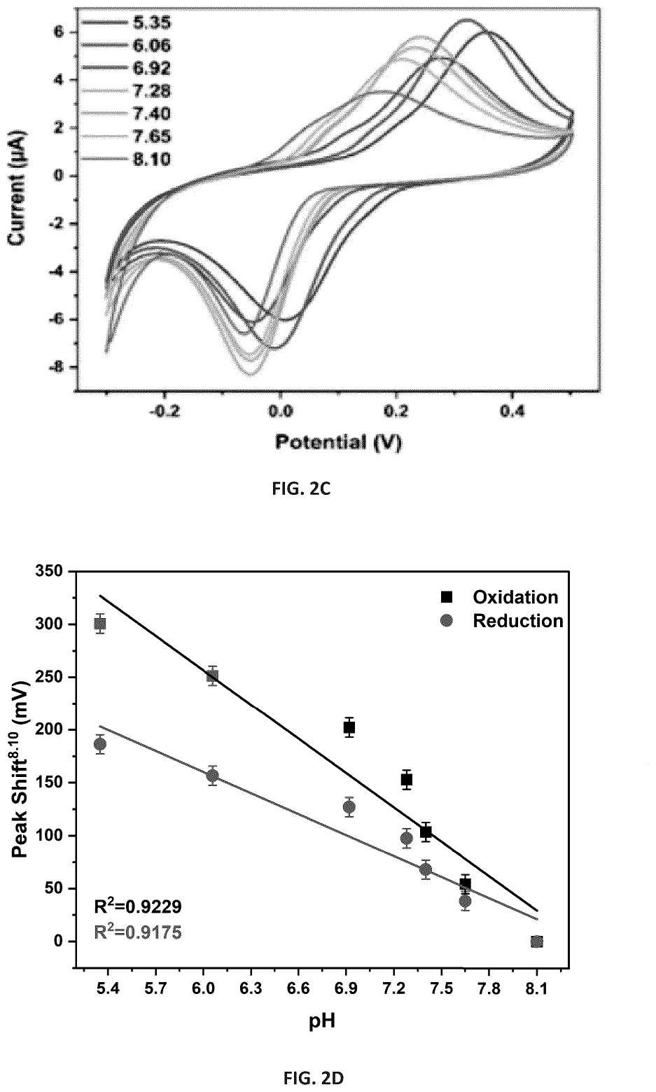

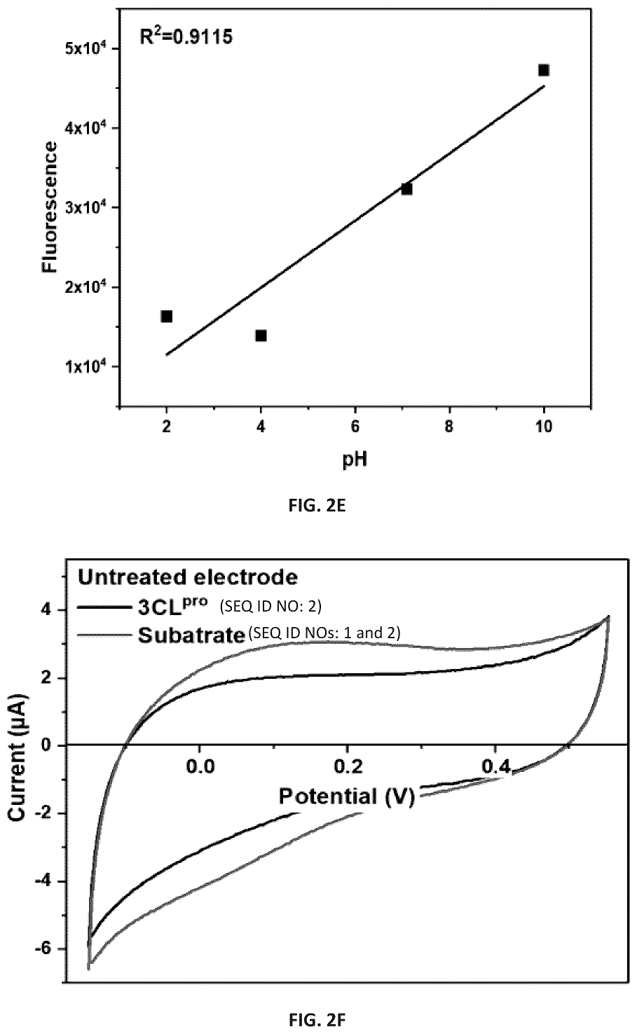

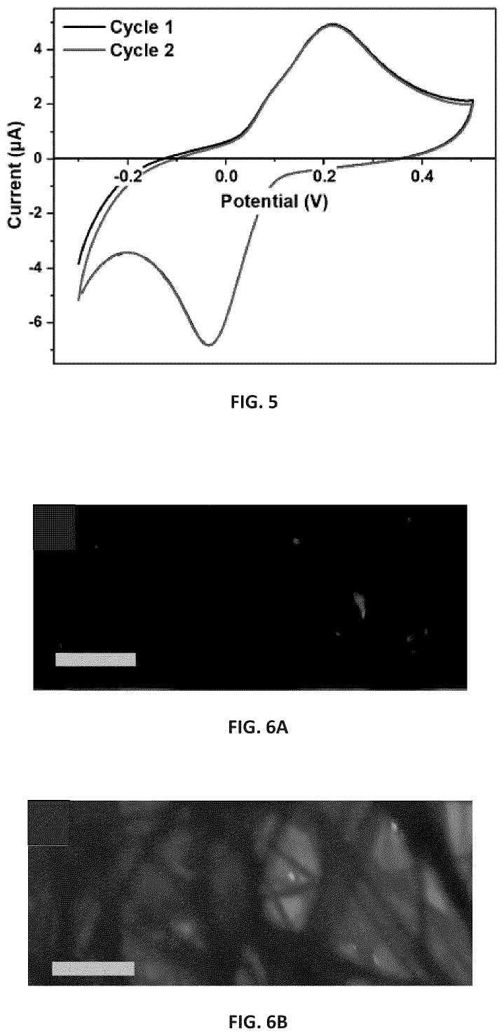

The present inventors have showed that the activity of an exemplary viral biomarker, SARS-CoV-2 3CL^{pro} (3CL^{pro}; SEQ ID NO: 2), can be quantified by measuring the pH change resulting from its proteolytic activity (FIG. 2A). This quantification revealed a pH plateaus at 8 minutes, which indicated a maximal required timeframe for detecting this viral biomarker, and allowed assessing ΔpH in the range of 0.35-0.74 following the presence of 3CL^{pro} in a solution with an initial pH of 7.4. This has led the present inventors to use a pH-dependent redox probe (p-benzoquinone (pBQ); (FIG. 2B)) as an exemplary electroactive substance that generates an electrochemically-detectable species, can serve as a pH-dependent redox probe. Peak shift (the difference between the voltage at maximal oxidation current after 2 minutes of CPE incubation in the sample and the voltage at maximal oxidation current after adding 3CL^{pro} substrate, SEQ ID NO: 1) was established as a reliable characterization parameter (Example 2 and FIGS. 2C-D, FIG. 5).

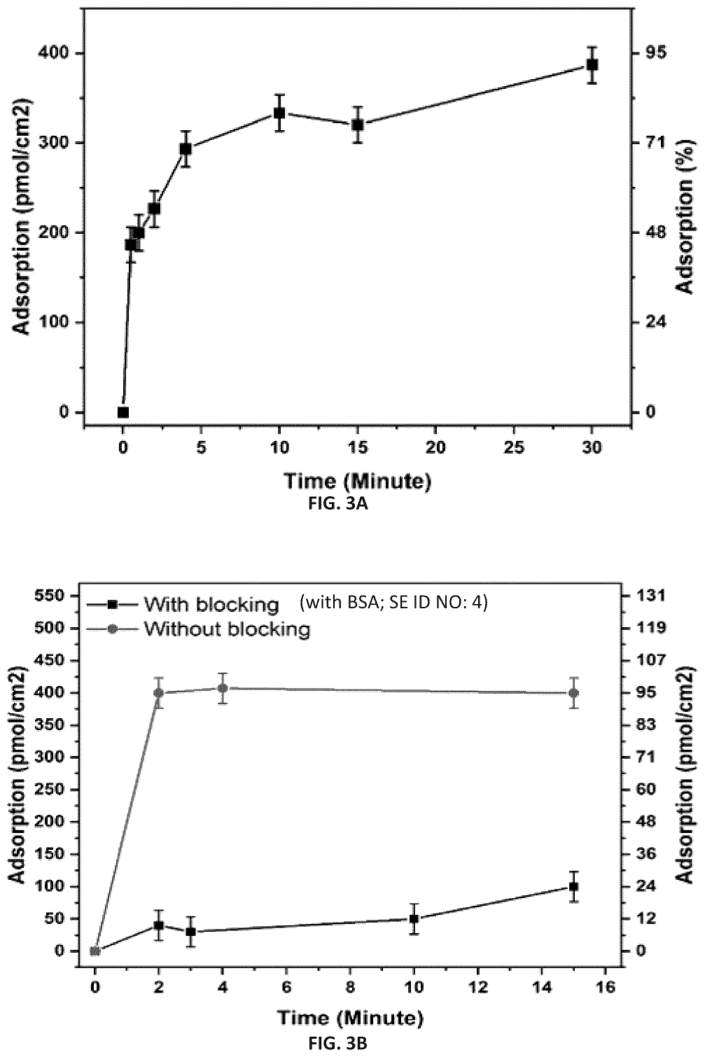

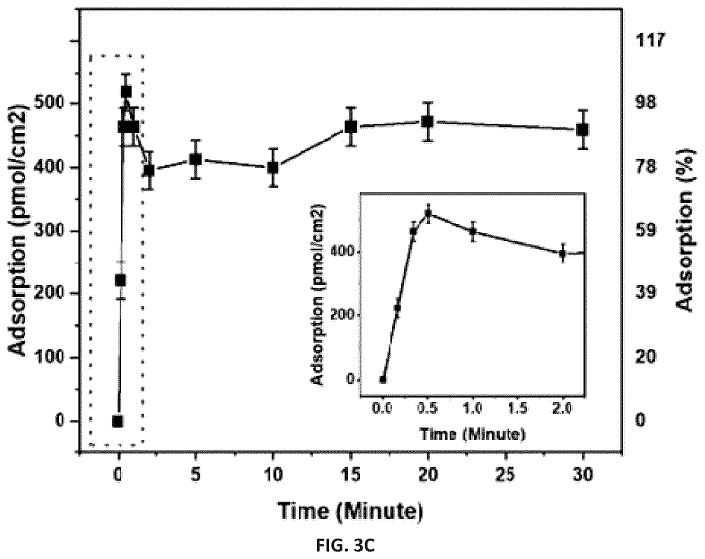

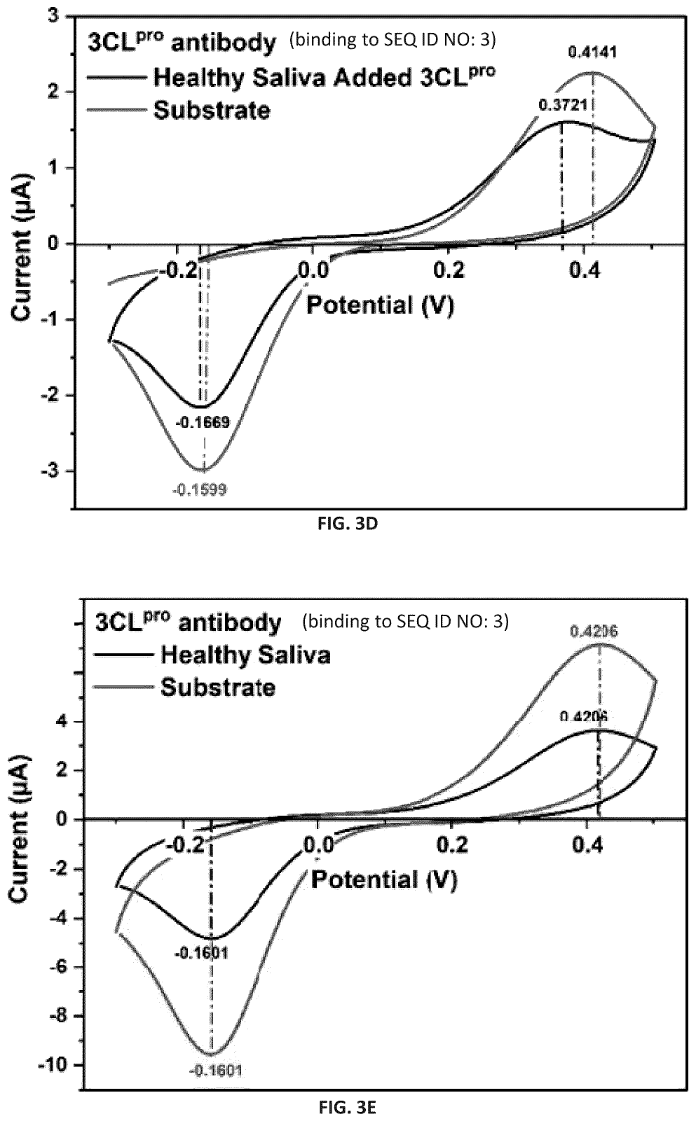

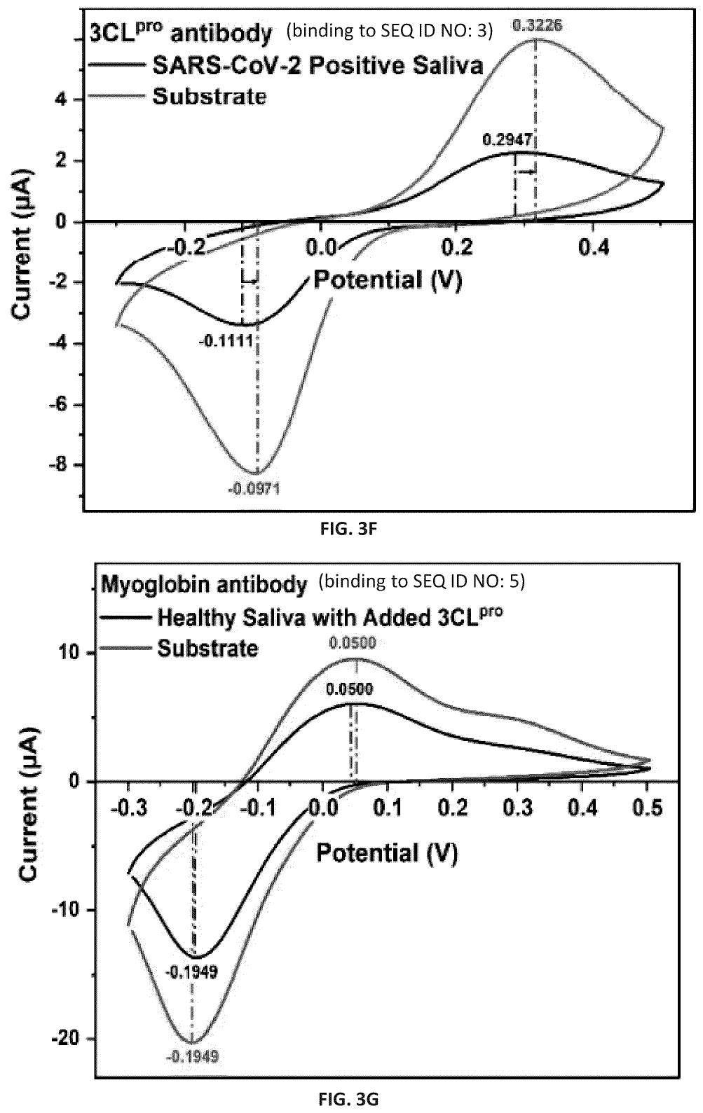

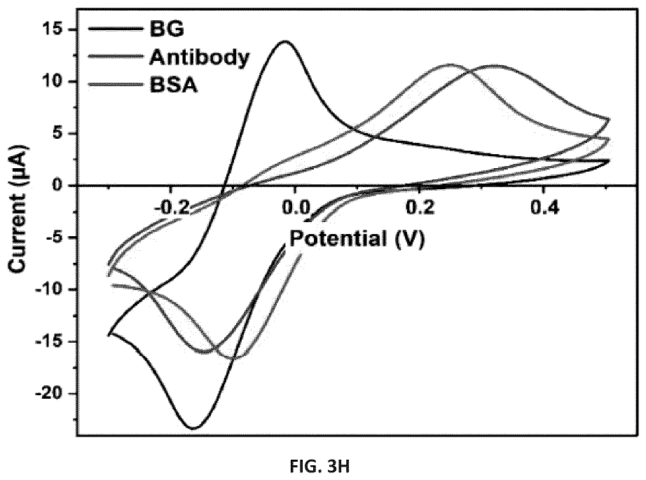

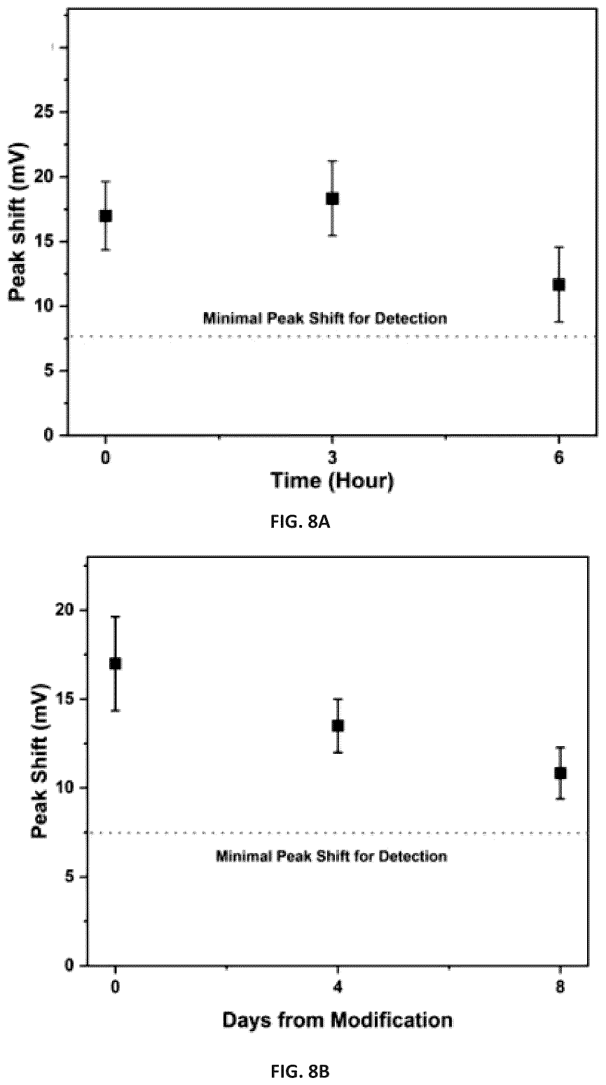

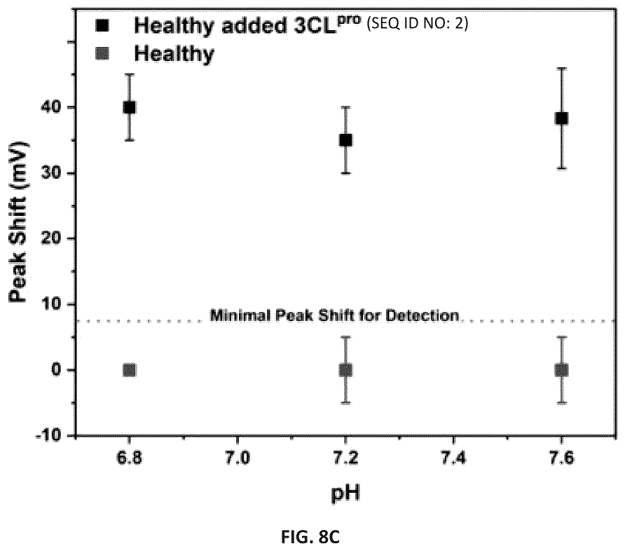

A successful detection of samples spiked with 3CL^{pro} (SEQ ID NO: 2) using this exemplary methodology has been demonstrated, as shown in FIGS. 3D-F and FIGS. 8A-C and Example 5.

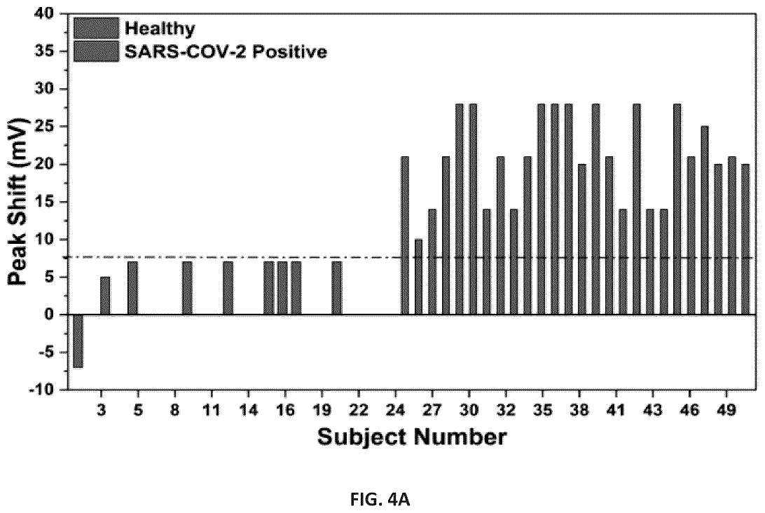

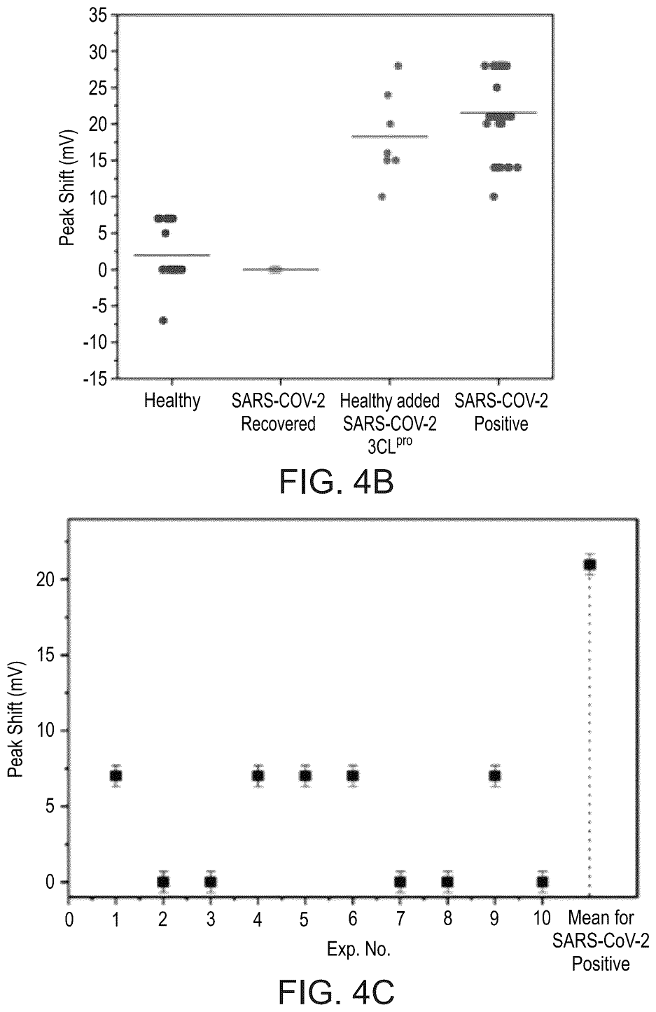

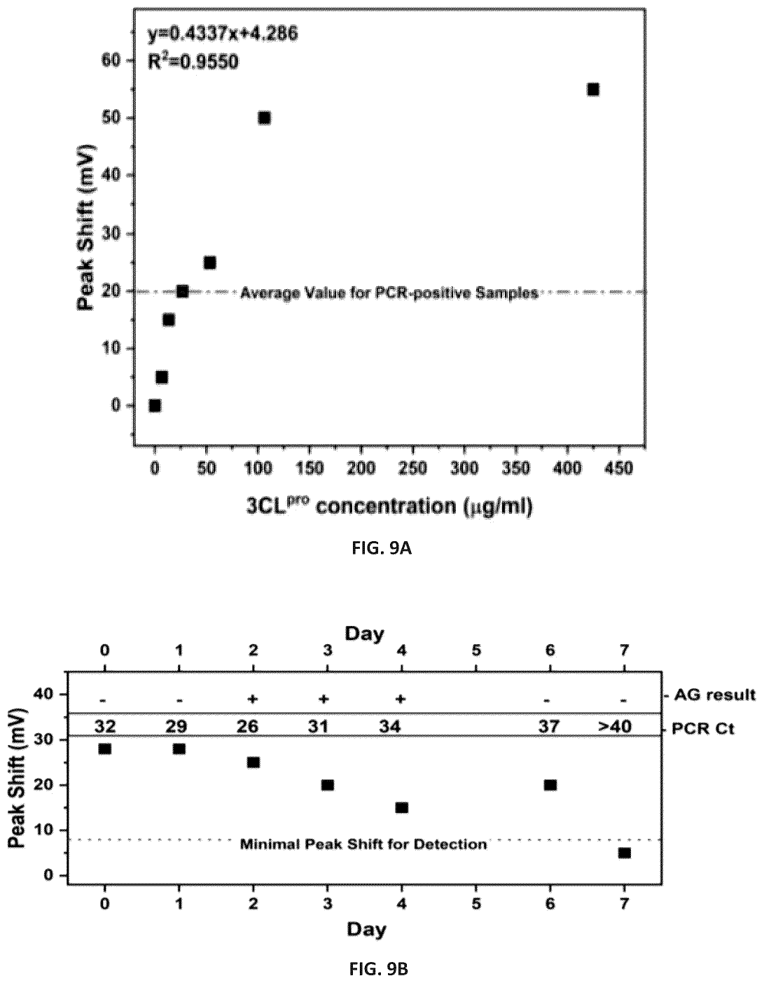

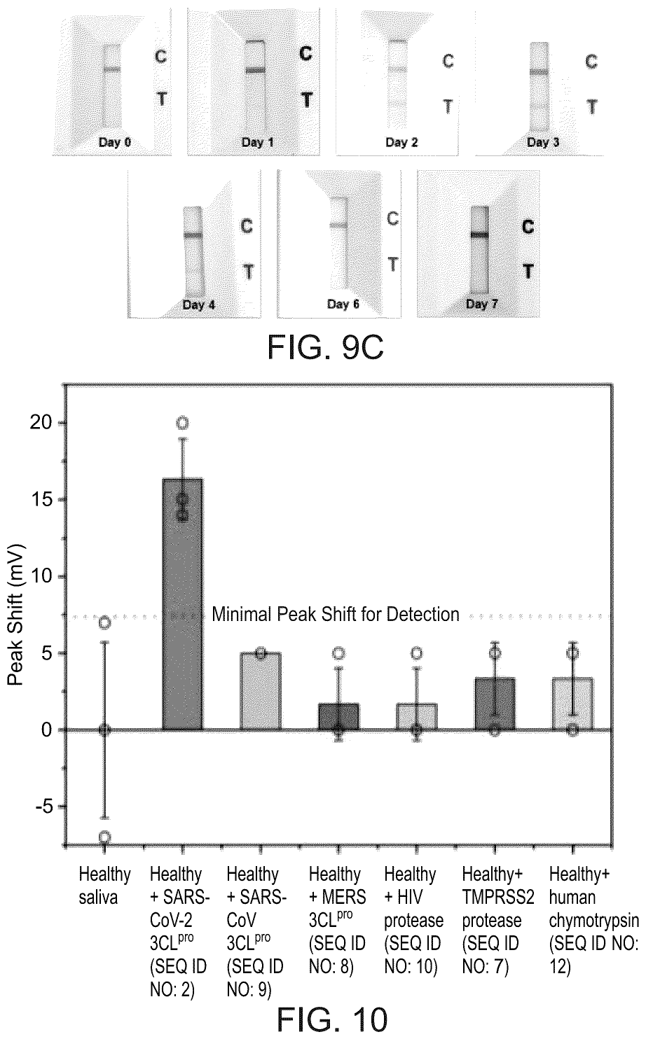

Clinical tests of saliva samples from 50 subjects (FIGS. 4A-C, FIGS. 9A-C and FIG. 10) showed accurate detection of SARS-CoV-2, with high sensitivity and specificity, validated by PCR testing of the samples. This is the first demonstration of 3CL^{pro} detection in saliva samples from SARS-CoV-2 patients as a specific indication of active SARS-CoV-2 infection. This platform displays the fastest detection turnover rate reported.

The clinical trial showed remarkable accuracy, specificity, and sensitivity, all these coupled with the ultrafast detection turnover, simplicity, low-cost and point-of-care compatibility of the platform, making it a promising method for the real-world SARS-CoV-2 mass-screening.

Embodiments of the present invention relate to an electrode having attached thereto an agent that specifically (selectively) binds to a viral biomarker, which is also referred to herein as an immune-functionalized electrode, to an electrochemical system comprising the immune-functionalized electrode, and to methods utilizing the immune-functionalized electrode or the system containing same in determining the presence and/or amount (level) of a respective virus.

Embodiments of the present invention relate to novel functionalized electrodes, to electrochemical systems containing same and to methods utilizing same for electrochemical detection of a viral infection such caused by SARS-CoV-2.

According to an aspect of some embodiments of the present invention there is provided an electrode (e.g., a carbon electrode such as a carbon paper electrode, preferably featuring high surface area) having attached thereto an agent that specifically binds to a viral biomarker. According to some embodiments, the viral biomarker is a proteolytic enzyme, for example, SARS-CoV-2-specific proteolytic enzyme, 3CL^{pro}, and the agent that specifically binds to the enzyme is a respective antibody, that is, 3CL^{pro}-specific antibody. Such an electrode is also referred to herein as immune-functionalized or an immuno-functionalized sensing electrode.

According to an aspect of some embodiments of the present invention there is provided an electrochemical system that comprises an electrode as described herein and an electrolyte. In some embodiments, the electrolyte comprises an electroactive agent that undergoes a redox reaction in response to an interaction between the viral biomarker and the agent that specifically bind it. In some embodiments, the interaction results in a pH change and the electroactive agent undergoes a pH-dependent redox reaction.

According to an aspect of some embodiments of the present invention there is provided an electrochemical system that comprises an electrode as described herein in any of the respective embodiments and any combination thereof.

According to an aspect of some embodiments of the present invention there is provided a method of determining a presence and/or amount of a viral infection in a subject, which is effected by contacting a biological sample drawn from the subject with the electrode or the electrochemical system as described herein in any of the respective embodiments and any combination thereof.

According to some embodiments of the present invention, the electrode, the system and/or the method as described herein are designed to determine a presence and/or amount of a corona virus, e.g., SARS-CoV-2, in a subject.

### Electrode:

According to an aspect of some embodiments of the present invention, there is provided an electrode having attached thereto an agent that specifically binds to a viral biomarker.

Herein, “an agent that specifically binds to a viral biomarker” is also referred to as a sensing agent or as a bioanalyte-specific agent or as a biomarker-specific agent.

According to some of any of the embodiments described herein, the electrode (e.g., electrode 100 as described herein) features a high surface area.

According to some of any of the embodiments described herein, the electrode (e.g., electrode 100 as described herein) features a surface area of at least 1000 m² gram⁻¹.

In some embodiments, the electrode is a porous electrode.

In some embodiments, the electrode comprises a high-surface area conductive or semi-conductive matrix (including, for example, carbon porous matrices and metal 3D porous matrices).

In some embodiments, the conductive (or semi-conductive) matrix is associated with nanostructures (e.g., nanowires, nanoparticles and/or nanotubes) for the formation of super-large area conductive composite electrodes.

In some embodiments, the conductive (or semi-conductive) matrix comprises biomolecular or polymeric species that can act as a chemical receptor/adsorption layer, in order to increase the adsorption characteristics of the electrode, and increase the adsorption of the pathogenic organism of the portion thereof from the tested sample to the electrode.

In some embodiments, the electrode comprises a carbon microporous or nanoporous 3D matrix. In some embodiments, the electrode has attached thereto functional moieties that can improve the absorption capability of the electrode.

According to some of any of the embodiments described herein, the electrode is a commercially available electrode or a costume-made electrode. In any case, the electrode can be used per se or can be pre-treated before being used (e.g., immune-functionalized) as described herein.

Such a pre-treatment can include, for example, cleaning the electrode by washing it with an organic and/or aqueous solvent, subjecting the electrode to plasma treatment and/or chemically modifying the electrode so as to feature functional groups on its surface, for example, functional groups as described herein for facilitating or improving the attachment (e.g., as described herein) of the sensing agent thereto.

According to some of any of the embodiments of the present invention, the electrode features at least one nanoscale or microscale dimension.

By “microscale dimension” it is meant that at least one dimension of the electrode is lower than 1 mm, or ranges from 0.1 micron to 900 microns.

By “nanoscale dimension” it is meant that at least one dimension of the electrode is lower than 1 micron, or ranges from 0.1 nanometer to 900 nanometers.

The nanoscale or microscale dimension depends on the shape of the electrode. If an electrode is generally shaped as a cylinder, the at least one dimension can be one or both of a length and a diameter of the electrode. If the electrode is generally shaped as a rectangular, the at least one dimension can be one or more of a length and a width of the electrode.

Electrodes featuring one or more microscale or nanoscale dimension are also referred to herein and in the art as microelectrodes.

According to some embodiments of the present invention, the electrode is a carbon electrode.

According to some embodiments of the present invention, the electrode is a carbon microelectrode.

Carbon electrodes or microelectrodes can be made of glassy carbon, screen-printed carbon, carbon films, carbon fibers, carbon paste and others.

According to some embodiments of the present invention, the carbon electrode is a carbon fiber electrode, or a carbon fiber microelectrode (also referred to herein as a micro-carbon-fiber electrode, or a micro CF electrode or a CF microelectrode).

A carbon fiber (CF) electrode is an electrode that comprises elementary carbon (e.g., graphite) shaped as a fibrous structure (e.g., a filament). Generally, but not necessarily, a CF electrode features a microscale or even nanoscale diameter or width, typically, but not limited to, in a range of from 5 to 200 microns, or 5 to 100 microns, or 5 to 50 microns or 5 to 20 microns.

Generally, but not necessarily, a CF electrode features a length (height) of from about 100 microns to about 50 mm, or from about 100 microns to about 1 mm, or from about 100 microns to about 800 microns, including any intermediate values and subranges therebetween. A CF electrode featuring such dimensions is a CF microelectrode.

In some embodiments the CF microelectrode further comprises a mechanical support or a protective layer (e.g., lamination) enveloping or surrounding at least a portion of the electrode, leaving a protruding tip of e.g., from 10 to 100 microns, of unsupported, exposed portion of the electrode (e.g., for contacting the sample).

The CF microelectrode can be a single-barrel or a multi-barrel electrode.

Any commercially available CF microelectrode can serve as a raw material for providing a CF microelectrode according to the present embodiments, upon generating on at least a part of its surface a functional moiety as described herein.

In some embodiments, a CF microelectrode is a carbon paper electrode.

According to some of any of the embodiments described herein, the electrode is a carbon fiber microelectrode.

According to some of any of the embodiments described herein, the electrode is a carbon paper electrode, for example, a carbon paper microelectrode.

According to some of any of the embodiments described herein, the carbon paper microelectrode is a porous carbon paper microelectrode.

According to some of any of the embodiments described herein, the carbon paper microelectrode is used per se, and in some embodiments, it is pre-treated as described herein in any of the respective embodiments.

In some embodiments, the electrode as described herein (e.g., a carbon paper or carbon fiber microelectrode) is electrically connectable to other parts of an electrochemical sensing system (e.g., as described herein), that is, it comprises, or is attachable to electrically conducting wires, for example, conducting metal foils such as Ni foils.

According to some of any of the embodiments described herein, the electrode, e.g., a CF microelectrode, has electrically conducting wires in electric communication therewith.

The electrode (e.g., electrode 100 as described herein) can alternatively be made of other carbon-containing configurations and/or other conductive materials or a mixture of conductive materials, preferably while featuring porosity and/or high surface area as described herein, and/or while allowing a biological sample or a portion thereof be absorbed to at least a part of its surface.

According to the present embodiments, the electrode has a sensing agent as described herein attached to at least a portion of the electrode. Such an electrode is also referred to herein as an immune-functionalized electrode, or a modified electrode, or electrode 102.

The sensing agent can be attached to the electrode chemically, e.g., by means of covalent attachment, electrostatic interactions, hydrogen bond interactions, aromatic interactions, etc., or physically (by being adsorbed to, entangled with, encapsulated in, or deposited on a surface or part thereof of, the electrode or a part thereof.

According to some of any of the embodiments described herein, the sensing agent is physically attached to the electrode or a part thereof, and in some embodiments, the sensing agent is adsorbed to the electrode.

According to some of the present embodiments, an electrode having a sensing agent attached (e.g., adsorbed) thereto as described herein is prepared by contacting the electrode with the sensing agent.

An exemplary procedure for preparing a carbon electrode having a sensing agent adsorbed to a portion thereof is described in the Examples section that follows.

According to some of any of the embodiments described herein, the electrode (e.g., electrode 102) further comprises, in addition to the sensing agent, an agent that interferes or inhibits attachment (e.g., as described herein, for example, physical attachment such as adsorption) to the electrode of proteins or other biological species other than the viral biomarker to be detected.

According to some of these embodiments, such an agent is or comprises a proteinaceous material that is incapable of interacting, or which has a weak and reversible interaction (high dissociation constant Kd), with biological species.

When such an agent is attached (e.g., adsorbed) to the electrode (e.g., electrode 102) subsequent to attaching the sensing agent, it occupies sites of the electrode that are free of the sensing agent, and thus reduces or prevents adsorption of biological species other than the viral biomarker once the electrode is contacted with a biological sample as described herein.

Any agent that may perform to reduce or present such an undesired adsorption is contemplated. Non-limiting examples include BSA and/or skimmed milk.

According to some embodiments, an electrode as described herein is prepared by contacting the electrode with the sensing agent, as described herein, optionally washing the electrode thereafter, contacting the electrode with the agent that interferes with binding of other biological species as described herein, for example, by soaking the electrode modified with the sensing agent in a solution that comprises this agent, and optionally washing the electrode thereafter, preferably with a buffer solution.

An exemplary procedure for preparing such a carbon electrode (e.g., electrode 102) is described in the Examples section that follows.

The electrode as described herein is designed to performed as a sensing electrode for determining a presence and/or amount of a viral biomarker (e.g., as electrode 102), as described herein.

By “viral biomarker” as used herein it is meant a biological species (e.g., a proteinaceous material such as an antigen, an enzyme, a cytokine), a nucleic acid material (e.g., RNA), or a small molecule (e.g., a metabolite) that is indicative of a presence of a viral infection, typically by being upregulated as a result of a viral infection. According to some embodiments, the viral biomarker is selected as being upregulated during an active viral infection in a subject.

Herein, the phrase “viral biomarker” is also referred to herein as a biomarker indicative of a viral infection, and in some embodiments, as indicative of an active viral infection in a subject.

The phrase “active viral infection” means that an active virus causing the viral infection is present in the subject.

An agent that specifically binds to a viral biomarker, which is also referred to herein as a biomarker-specific agent or a biomarker-specific reagent, or simply as a sensing agent, describes an agent that binds to the viral biomarker at a much higher level than to another, even structurally or functionally similar, species, e.g., biological species. In some embodiments, this agent is such that its binding affinity to the viral biomarker is characterized by a dissociation constant, Kd, of no more than 1 mM, or no more than 100 nM, or no more than 10 nM, or no more than 1 nM, or no more than 10⁻¹⁰M, or no more than 10⁻¹²M, and even lower, e.g., as low as 10⁻¹⁵M.

The interaction between the selected agent and the viral biomarker can be reversible or irreversible.

In some of any of the embodiments described herein, the viral biomarker and the respective agent form an affinity pair, as defined herein.

In some embodiments, the agent is a bioanalyte specific reagent, as defined by the FDA (see, (ASRs) in 21 CFR 864.4020).

In some embodiments, the biomarker and its respective specific agent form an affinity pair, characterized by a dissociation constant, K_{D} lower than 10⁻⁵ M, or lower than 10⁻⁷ M, or lower than 10⁻⁸ M, than 10⁻¹, or than 10⁻¹⁰ M.

Exemplary affinity pairs include, without limitation, an enzyme-substrate pair, a polypeptide-polypeptide pair (e.g., a hormone and receptor, a ligand and receptor, an antibody and an antigen, two chains of a multimeric protein), a polypeptide-small molecule pair (e.g., avidin or streptavidin with biotin, enzyme-substrate), a polynucleotide and its cognate polynucleotide such as two polynucleotides forming a double strand (e.g., DNA-DNA, DNA-RNA, RNA-DNA), a polypeptide-polynucleotide pair (e.g., a complex formed of a polypeptide and a DNA or RNA e.g., aptamer), a polypeptide-metal pair (e.g., a protein chelator and a metal ion), a polypeptide and a carbohydrate (leptin-carbohydrate), and the like.

In exemplary embodiments, the agent that specifically binds the viral biomarker is an antibody specific to the viral biomarker.

According to some of any of the embodiments described herein, the viral biomarker is a proteolytic enzyme (e.g., a protease), which is upregulated (e.g., overexpressed and/or overactive) during a viral infection.

According to some of any of the embodiments described herein, the agent that specifically or selectively binds to the biomarker is an antibody specific to the proteolytic enzyme.

Herein throughout, the terms “specifically” and “selectively” are used interchangeable.

According to some of any of these embodiments, the antibody binds to the enzyme in such a way that does not affect its enzymatic activity. In some embodiments, the antibody binds to a certain sequence of amino acids of the enzyme and this binding does not affect chemically and/or sterically the catalytic binding site of the enzyme.

According to an aspect of some embodiments of the present invention, the viral biomarker is such that is present in a saliva of a subject having a viral infection as described herein, and the sensing agent is selected selective to such a viral biomarker. This allows determining a presence of a viral infection by contacting a saliva sample of the subject with the electrode.

According to some of any of the embodiments described herein, the sensing agent is selected such that its interaction with the viral biomarker generates, directly or indirectly, via subsequent steps and/or reactions, an electrochemically-detectable species or moiety, as described in further detail hereinafter.

According to some of any of the embodiments described herein, the sensing agent is such that binds to the viral biomarker without affecting (e.g., reducing or inhibiting) its activity.

According to some of any of the embodiments described herein, the viral biomarker is an enzyme, and in some embodiments it is a proteolytic enzyme, which is indicative of the viral infection, as described herein, for example, is upregulated in a subject having a viral infection, preferably an active viral infection.

Determining a presence of an enzymatic biomarker is advantageous as it allows determining electrochemically an interaction of the enzyme with its substrate, while requiring only catalytic amounts of the enzyme for generating a detectable amount of electrochemically-detectable species or moieties.

Determining a presence of a proteolytic enzyme is further advantageous, since proteolysis of a respective substrate typically generates species such as protons that can be readily detected electrochemically.

According to some of any of the embodiments described herein, the agent that specifically binds to the viral biomarker is an antibody specific to viral biomarker.

According to some of any of the embodiments described herein, the antibody is such that binds to the viral biomarker without affecting (e.g., reducing or inhibiting) its activity.

According to some of any of the embodiments described herein, the antibody is specific/selective to an enzyme, for example, a proteolytic enzyme, which is indicative of the viral infection, and is preferably upregulated as result of the viral infection (e.g., an active viral infection).

Preferably, the antibody is such that binds to the enzyme without affecting its catalytic activity. For example, the antibody is selected such that when it is bound to the enzyme, it does not affect chemically or does not sterically hinder, an interaction between the enzyme and its substrate. In some embodiments, the antibody binds a region of the enzyme that is other than the catalytic binding site of the enzyme and which does not hinder sterically an interaction between the enzyme and its substrate.

According to some of the present embodiments, the electrode is usable in determining a presence and/or amount/level of a viral biomarker and is therefore usable in determining a presence and/or amount/level of a viral infection. Accordingly, a sensing agent that selectively binds to the viral biomarker is selected in accordance with biomarkers indicative of a viral infection to be determined or detected.

The viral infection to be detected while using an electrode as described herein, can be caused by any virus (a viral pathogen).

Non-limiting types of viral pathogens that cause viral infections include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Non-limiting examples of viral infections include human immunodeficiency virus (HIV)-induced acquired immunodeficiency syndrome (AIDS), coronavirus, influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

According to specific embodiments, the disease is a Coronavirus infection.

According to specific embodiments, a clinical manifestation of Coronavirus infection includes symptoms selected from the group consisting of inflammation in the lung, alveolar damage, fever, cough, shortness of breath, diarrhea, organ failure, pneumonia and/or septic shock.

As used herein, “Coronavirus” refers to enveloped positive-stranded RNA viruses that belong to the family Coronaviridae and the order Nidovirales.

Examples of Corona viruses which are contemplated herein include, but are not limited to, 229E, NL63, OC43, and HKU1 with the first two classified as antigenic group 1 and the latter two belonging to group 2, typically leading to an upper respiratory tract infection manifested by common cold symptoms.

However, Coronaviruses, which are zoonotic in origin, can evolve into a strain that can infect human beings leading to fatal illness. Thus particular examples of Coronaviruses contemplated herein are SARS-CoV, Middle East respiratory syndrome Coronavirus (MERS-CoV), and SAR-CoV-2 [causing 2019-nCoV (also referred to as “COVID-19”)].

It would be appreciated that any Coronavirus strain is contemplated herein even though SAR-CoV-2 is emphasized in a detailed manner.

According to specific embodiments, the viral infection is a SAR-CoV-2 infection and the viral biomarker is indicative of SAR-CoV-2 infection or to the presence of a SAR-CoV-2 virus in a subject, and is also referred to herein as a SAR-CoV-2 biomarker.

According to some of any of these embodiments, the SAR-CoV-2 biomarker is such that is present in a saliva of a subject having a SAR-CoV-2 infection.

According to some of any of these embodiments, the viral biomarker is a SARS-CoV-2-specific proteolytic enzyme.

According to some of any of these embodiments, the viral biomarker is a SARS-CoV-2-specific proteolytic enzyme that is present in the saliva of a subject having a SARS-CoV-2 infection.

According to some of any of these embodiments, the agent that specifically binds to the SARS-CoV-2-specific proteolytic enzyme is an antibody specific to the SARS-CoV-2-specific proteolytic enzyme.

According to some of any of these embodiments, the SARS-CoV-2-specific proteolytic enzyme is 3CL^{pro} (SARS-CoV-2 3CL^{pro}). According to some of these embodiments, the agent that specifically binds to the biomarker is an antibody specific to said SARS-CoV-2 3CL^{pro}.

An exemplary SARS-CoV-2 3CL^{pro} is such that has or comprises an amino acid sequence as set forth in SEQ ID NO: 2.

As used herein throughout, “3C-like protease”, which is also referred to herein simply as 3CL protease or 3CL^{pro}, describes an enzyme identified by the EC number EC 3.4.22.69. While the amino acid sequence of 3CL^{pro} is typically conserved, a wild-type 3CL^{pro} enzyme can be 3CL^{pro} of a mammal (e.g., human, rabbit) or of any other organism, including microorganisms (e.g., virus).

An amino acid sequence of an exemplary SARS-CoV-2 3CL^{pro}, an E. coli-derived SARS-CoV-2 3CL^{pro o}, is set forth in SEQ ID NO: 2. A 3CL^{pro} enzyme as referred to herein is homologous to SEQ ID NO: 2 by at least 50%, or at least 60%, or at least 70%, or at least 80%, or at least 90%, or can be 100%, homologous to SEQ ID NO: 2.

By “wild-type” it is meant the typical form of the enzyme as it occurs in nature, e.g., in an organism or microorganism. A wild-type 3CL^{pro} enzyme encompasses an enzyme isolated from an organism or a microorganism, a chemically synthesized enzyme, and a recombinantly prepared enzyme.

According to some of any of the embodiments described herein, the electrode (e.g., electrode 102) has attached thereto an antibody that is selected to bind selectively to a proteolytic enzyme, for example to 3CL protease, such as described herein in any of the respective embodiments. Such antibodies, or fragments thereof, can be prepared using methods well-known in the art, and some are commercially available.

According to some of any of the embodiments described herein, the antibody binds to a portion of the amino acid sequence as set forth in SEQ ID NO: 2 or in SEQ ID NO: 3, such that the binding does not affect the catalytic activity of the enzyme.

According to exemplary embodiments, such a portion of a SARS-CoV-2 3CL^{pro} (e.g., which has or comprises the amino acid sequence as set forth in SEQ ID NO: 2) has an amino acid sequence as set forth in SEQ ID NO: 3.

According to exemplary embodiments, the antibody is selected as such that selectively binds to SARS-CoV-2-3CL^{pro} antigen having an amino acid sequence as set forth in SEQ IS NO: 2 or 3.

Antibodies, or fragments thereof, which selectively bind to a selected portion of an enzyme as described herein can be produced by methods known in the art, and are sometimes commercially available.

Exemplary commercially available antibodies that are selective to SARS-CoV-2 3CL^{pro} include, but are not limited to, antibodies available from Novus Biologicals® (Rabbit-derived SARS-CoV-2 3CL Protease Antibodies NBP3-07061, NBP3-07062, NBP3-13458, NBP3-13468); SARS-CoV-2 3CL Protease Antibody PA5-116940), Thermo Fisher Scientific® (Invitrogen rabbit-derived SARS-CoV-2 3CL^{pro} Polyclonal Antibody #PA5-116940) and Cell Signaling Technology® (rabbit-derived SARS-CoV-2 3C-Like Protease Antibody #51661).

The term “antibody” as used herein includes intact molecules as well as functional fragments thereof, such as Fab, F(ab′)2, and Fv that are capable of binding to the indicated biomolecule (e.g., biomarker). These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368: 856-859 (1994); Morrison, Nature 368 812-813 (1994); Fishwild et al., Nature Biotechnology 14, 845-51 (1996); Neuberger, Nature Biotechnology 14, 826 (1996); and Lonberg and Huszar, Intern. Rev. Immunol. 13, 65-93 (1995).

### Electrochemical System and Method:

According to some of the present embodiments, the electrode as described herein (e.g., sensing electrode 102), is usable in determining a presence and/or level or amount of a viral biomarker in a sample, as described herein, and hence also for determining a presence and/or level or amount of a viral infection in a subject (e.g., a subject suspected as having the viral infection).

According to some of any of the embodiments described herein, an electrode as described herein (e.g., electrode 120) is usable in the methods and uses as described herein, upon being contacted with a sample integrated in an electrochemical system (also referred to herein as a sensing system or a part thereof).

According to an aspect of some embodiments of the present invention, there is provided an electrochemical system that comprises an electrode as described herein in any of the respective embodiments, having attached thereto a sensing agent as described herein.

Herein, electrode (e.g., electrode 102), having a sensing agent attached thereto is contacted with a sample as described herein, to thereby provide electrode 120.

According to some of any of the respective embodiments, the electrochemical system is configured so as to generate, directly or indirectly (following a sequence of steps and/or reactions), a detectable change is an electrochemical parameter upon contacting the electrode with the viral biomarker, as a result of an interaction of the viral biomarker with the sensing moiety.

According to some of any of the embodiments described herein, the electrode (e.g., electrode 120) is integrated or forms a part of an electrochemical cell.

According to some of any of the embodiments described herein, the electrode (e.g., electrode 120) forms a part of an electrochemical cell and the electrochemical cell is operable by electrically connecting the electrode (e.g., electrode 120) to a power source.

In some embodiments of the present invention, there is provided an electrochemical cell which comprises a sensing electrode as described herein in any of the respective embodiments and any combination thereof (e.g., electrode 120). The sensing electrode functions, and is also referred to herein, as a working electrode.

In some embodiments of the present invention, there is provided a sensing system which comprises an electrochemical cell as described herein in any of the respective embodiments and any combination thereof.

The following describes some embodiments of an electrochemical cell of the invention and the method of operating it.

In some embodiments, the sensing electrode is electrically connectable to a power source, as described herein, and the cell is configured such that when it is operated, at least a portion thereof contacts a solution (an electrolyte solution; e.g., electrolyte 18) that comprises at least agent 122 as described herein in any of the respective embodiments.

In some embodiments of the present invention, the electrochemical cell further comprises a reference electrode (e.g., electrode 22). Any commercially available or customarily designed reference electrode is contemplated. In some embodiments, the reference electrode is an aqueous reference electrode. Exemplary usable reference electrodes include, but are not limited to, Silver/Silver Chloride electrode (e.g., Ag/AgCl/Saturated KCl electrode such as marketed by Metrohm), a Standard calomel (e.g., saturated calomel) electrode (SCE), a Standard hydrogen electrode (SHE), a Normal hydrogen electrode (NHE), a Reversible hydrogen electrode (RHE), a Copper-copper(II) sulfate electrode (CSE); a pH-electrode; a Palladium-hydrogen electrode, a Dynamic hydrogen electrode (DHE), and a Mercury-mercurous sulfate electrode (MSE).

The reference electrode is also electrically connectable to a power source, and the cell is configured such that when it is operated, a potential difference (voltage) is applied between the sensing electrode (e.g., electrode 120) and the reference electrode (e.g., electrode 22).

In some embodiments, the electrochemical cell follows a three-electrode design and further comprises an auxiliary electrode. Preferably, but not obligatory, the auxiliary electrode is a platinum electrode. Any other auxiliary electrode, commercially available or customarily designed, is contemplated. Non-limiting examples include gold electrodes, carbon electrodes and carbon/gold electrodes.

In some embodiments, the auxiliary electrode is electrically connectable to the sensing electrode, for example, electrically-conductive wires connect the electrodes.

In some embodiments, the electrochemical cell further comprises a device that measures a current generated at the sensing electrode, as a result of electrochemically-detectable (e.g., redox) reactions occurring at or next to a surface of the sensing electrode. In some embodiments, this device (e.g., an amperometer, a picoameter) is electrically connectable to the auxiliary electrode and the sensing electrode.

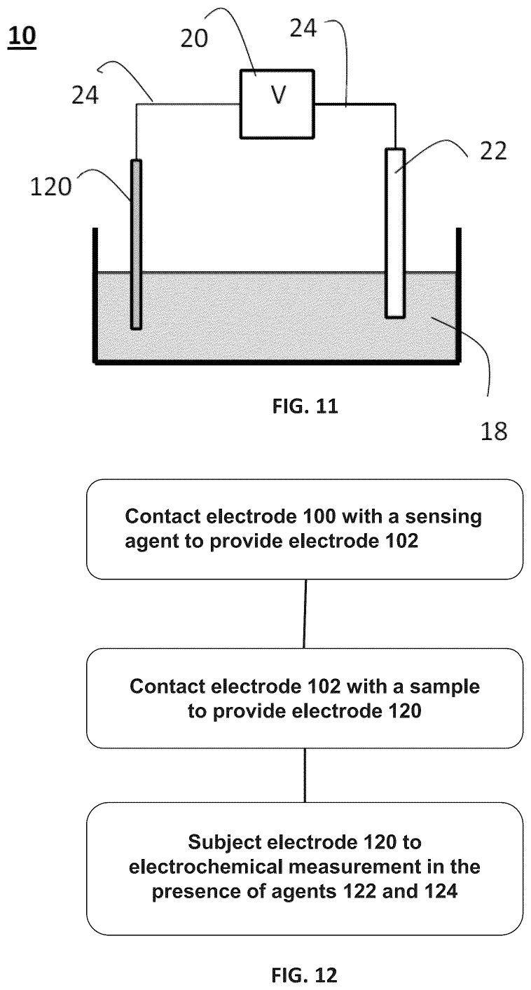

A schematic presentation of an exemplary assembly of a two-electrode electrochemical cell according to some embodiments of the present invention is presented in FIG. 11.

Electrochemical cell 10 comprises a sensing electrode 120 as described herein, which acts as a working electrode. When the cell is operated, electrode 120 should be in contact with an electrolyte 18 which comprises at least agent 122. Sensing electrode 120 is one half of electrochemical cell 10. A reference electrode 22 is the other half of cell 10. A power source 20 is electrically connectable or connected to sensing electrode 120 and reference electrode 22 by means of electrical wires 24. Power source 20 is configured to apply voltage between sensing electrode 120 and reference electrode 22. Optionally, but not obligatory, cell 10 further comprises an auxiliary electrode (not shown), and a current measuring device 28, and device 28 is electrically connectable or connected to sensing electrode 120 and auxiliary electrode 26.

For an electrochemical cell (e.g., cell 10) to operate, at least the sensing electrode (electrode 120) should be in contact with an electrolyte shown in FIG. 11 as an electrolyte 18. The electrochemical cell (e.g., cell 10) can comprise an electrolyte (e.g., electrolyte 18, as exemplified in FIG. 11), or can comprise means (e.g., an inlet port; not shown in FIG. 11), for introducing the electrolyte to the cell, so as to contact at least the sensing electrode (e.g., sensing electrode 120).

An electrochemical cell according to the present embodiments can follow any of the designs known in the art, and can include one or more sensing electrodes, and one or more of a reference electrode and/or an auxiliary electrode. Exemplary designs include, without limitation, rotating disk-ring electrodes, ultramicro-electrodes, or screen printed electrodes.

The configuration of the components of electrochemical cell 10 as presented in FIG. 11 are for illustrative purpose only and are not to be regarded as limiting in any way.

Electrochemical cell 10 can be, for example, in a form of a covered glass (or other inert material like Teflon or quartz) beaker, containing the sample solution in which the three electrodes are dipped. In some embodiments, electrochemical cell 10 is a micro cell or a thin layer cell.

Electrochemical cell 10 may further comprise means for mixing/stirring electrolyte 18 and agent 122 or any other agents included in the electrolyte (not shown in FIG. 11).

Electrochemical cell 10 may further comprise means for monitoring and/or controlling the temperature inside the cell (not shown in FIG. 11).

As used herein and in the art, an electrolyte is an electrically conducting material or medium. An electrolyte can be solid or fluid, and can be used per se or when dissolved in a polar solvent, such as water. When dissolved is a solvent, it is referred to as an electrolyte solution. In the context of electrochemical cells, an electrolyte is also referred to as a background solution.

Herein throughout, the term “electrolyte” also encompasses an “electrolyte solution”.

In an electrochemical cell as described herein (e.g., cell 10, FIG. 11), at least the sensing electrode (e.g., sensing electrode 120) contacts the electrolyte (e.g., electrolyte 18) when the cell is operated. In some embodiments, all electrodes contact an electrolyte (e.g., electrolyte 18) when the cell is operated. In some embodiments, all electrodes contact the same electrolyte, as exemplified in FIG. 11, and in some embodiments, one or more of the electrodes contact an electrolyte different from the electrolyte in contact with the sensing electrode, and a membrane is interposed between the different electrolytes.

The electrolyte (e.g., electrolyte 18) comprises a substance (e.g., substance 122) that is capable of interacting (e.g., selectively) with the viral biomarker, so as to generate, directly or indirectly, a detectable change is an electrochemical parameter in response to an interaction between the viral biomarker and the substance.

The substance (e.g., agent 122) is selected in accordance with the selected viral biomarker. Preferably, the substance (e.g., agent 122) is selected so as to generate, upon interacting with the viral biomarker, an electrochemically-detectable species or moiety.

In exemplary embodiments, the viral biomarker is a proteolytic enzyme and the substance (e.g., agent 122) is a substrate of the proteolytic enzyme.

According to some of any of the embodiments described herein, the electrolyte solution comprises a buffer that is suitable for performing the reaction between the viral biomarker and the substance (e.g., agent 122), e.g., the enzyme's substrate. For example, if agent 122 is an enzyme's substrate, the electrolyte solution (e.g., electrolyte 18) comprises a buffer or any other solution that features a pH at which the enzymatic catalysis is enabled. Similarly, the electrolyte solution is such that does not react with, or affects the stability of, agent 122.

According to exemplary embodiments, electrode 120 has adsorbed thereto a SARS-CoV-2 proteolytic enzyme, as described herein in any of the respective embodiments and any combination thereof, and electrolyte 18 comprises as agent 122 a substrate of the enzyme, for example, comprising or having SEQ ID NO:1. Any other available substrates of SARS-CoV-2 proteolytic enzyme (e.g., 3CL^{pro}) are contemplated (e.g., substrates having SEQ ID NOs: 16-36). Exemplary substrates are available from available synthetic peptide vendors.

The interaction between the viral biomarker and agent 122 can be an electrochemically-detectable reaction by itself, that is, it generates an electrochemically-detectable species or moiety directly (e.g., by being a redox reaction), or, it can generate a moiety or species that further interact (e.g., with a redox reactive substance such as agent 124) to generate electrochemically-detectable species or moiety.

When electrode 120 is subjected to an electrochemical reaction, an electric signal generated by this reaction, or by a sequence of reactions, means that electrode 120 has the viral biomarker associated therewith (attached thereto, e.g., adsorbed thereto), which means that the sample contained the viral biomarker to be detected or a portion thereof.

If no electric signal is generated, it means that the sample did not contain the viral biomarker or a portion thereof.

In some embodiments, the electric signal is a change of a background electric signal of the electrochemical cell or system.

Electrode 120 as described herein in any of the respective embodiments, is also referred to herein as “sensing electrode”, which can be subjected to electrochemical measurement/detection/sensing, preferably when integrated in an electrochemical cell or a system as described herein in any of the respective embodiments.

Once electrode 120 is generated (after contacting a sample) and optionally being washed, e.g., as described herein), electrode 120 is subjected to an electrochemically detectable reaction, as described herein. In some of these embodiments, electrode 120 can be contacted with an electrochemically detectable agent 122, as described herein in any of the respective embodiments and any combination thereof, preferably with a solution containing agent 122, that is, an electrolyte (e.g., electrolyte 18) that comprises agent 122, and electrochemical measurement is performed.

By “electrochemical reaction” it is meant a chemical reaction that involves a change in the electronic state of one or more substances that participate in the reaction, that is, acceptance or donation of electrons, which occurs in response to potential application.

By “electrochemical measurement” it is meant applying a potential to the electrode, and measuring an electric parameter in response to the potential application. If a change in the electric parameter occurs in response to potential application, the electrochemical measurement is indicative of a presence of an electrochemical reaction, and thereby of a presence of an electrochemically reactive substance.

By “electrochemically detectable reaction” it is meant a reaction that can be detected by electrochemical measurement, namely, a reaction that can be detected by a change of an electric parameter in response to potential application, that is, a reaction that produces and/or consumes an electrochemically detectable substance, species or moiety, as described herein.

By “electrochemically reactive substance” or “electroactive substance or agent” it is meant a substance that generates (donates) electrons or accepts (consumes) electrons in response to potential application.

An electrochemically reactive substance is typically a redox reactive substance, that undergoes reduction or oxidation in response to application of a potential lower than 5 Volts, or lower than 3 Volts, or lower than 2 Volts.

By “electrochemically detectable species or moiety” it is meant an electrochemically reactive species or moiety as described herein or a substance that produces or consumes an electrochemical reactive species or moiety.

Further description of embodiments pertaining to an electrochemically detectable reaction, and an electrochemically detectable species or moiety are provided hereinunder.

According to the present embodiments, there is provided a method of determining a presence and/or amount or level of a viral biomarker in a sample, which is effected as described herein. An exemplary flow chart of the method is presented in FIG. 12.

The method begins by preparing sensing electrode 102 by contacting electrode 100 as described herein with a sensing agent, to thereby product electrode 102, as described herein in any of the respective embodiments.

Electrode 102 can be prepared immediately prior to us, or can be prepared several minutes, hours or days, prior to us.

Thus, according to some embodiments, the method begins by contacting electrode 102 with a sample as described herein, to thereby obtain electrode 120.

Electrode 120 is thereafter integrated in an electrochemical cell or system (e.g., cell 10), and electrochemical measurements are performed by operating the electrochemical cell as described herein.

In some embodiments, the electrochemical reaction or detection is effected by contacting a sensing electrode as described herein in any of the respective embodiments (e.g., electrode 120) with a solution (e.g., electrolyte 18) that comprises agent 122, as described herein, and applying a potential to the sensing electrode.

In some embodiments, the electrochemical reaction or detection is further effected by measuring an electrochemical parameter upon applying the potential to the sensing electrode (electrode 120), and in some embodiments, the electrochemical parameter is an electrical current generated at the sensing electrode or a change in the electrical current at the sensing electrode. As described herein, a presence and/or level of the electrochemical parameter or of the change in the electrochemical parameter is indicative of a presence and/or level of the viral biomarker in the sample.

In some embodiments, the sensing electrode forms a part of an electrochemical cell (e.g., cell 10) as described herein in any of the respective embodiments, or a part of a sensing system as described herein in any of the respective parameters, and in some embodiments, contacting the sensing electrode (electrode 120) with agent 122 is effected by introducing the electrode to an electrochemical cell or system (e.g., cell 10), or integrating the electrode with the electrochemical cell or system as described herein (e.g., cell 10), that comprises agent 122 in a solution as described herein (e.g., with an electrolyte solution such as electrolyte 18 that comprises agent 122).

In some embodiments, applying a potential to the sensing electrode is performed after contacting the sensing electrode (e.g., electrode 120) with agent 122 or a solution containing same (e.g., with an electrolyte solution 18 that comprises agent 122).

In some embodiments, the sensing electrode is integrated to form a part of an electrochemical cell as described herein (e.g., cell 10) and applying the potential is performed by applying a voltage between the sensing electrode (e.g., electrode 120) and a reference electrode (e.g., electrode 22).

In some embodiments, the potential is a varying potential.

In some embodiments, measuring an electrochemical parameter is by voltammetry.

Voltammetry measurements are also referred to in the art as potentiostatic electrochemical analyses.

As known in the art, voltammetry experiments are conducted for obtaining information (e.g., presence, identity and/or level) of an analyte by measuring a generated current or a change in the current in response to application of a varying potential.

In order to obtain a quantitative measurement of an analyte (e.g., a redox reactive substance produced or consumed by the electrochemically detectable reaction) by potentiostatic electrochemical analysis, the amount of electrons used for the reduction/oxidation of the analyte should be monitored. In thermodynamic equilibrium the ratio of the redox-reactive species at the surface of the electrode can be obtained by Nernst equation:

$$E = {E}^{0} + \frac{2.3 R T}{nF} log ( \frac{{C}_{O}}{{C}_{R}} )$$

Where C_{O} is the concentration of the oxidized form, and C_{R} is the concentration of the reduced form, E is electrode potential, E⁰ is standard electrode potential, R is the gas constant (8.314J/Kmol) T is the temperature (Kelvin scale), n is the number of electrons participate in the redox reaction and F is the Faraday constant (96,487 coulombs).

The entire measured current is composed of Faradic currents and non-Faradaic charging background current. The Faradic current obtained by the electrochemical reaction behaves according to Faraday's low, which means that 1 mole of redox active substance will involve a charge change of n×96,487 coulombs.

The information retrieved by voltammetry experiments, in its simplest form, is obtained as a voltammogram of I=f(E).

A voltammogram is a current versus potential curve used to describe the analyte's electrochemical reaction performed at the electrode as a result of the applied potential, and its derived current. It may have a complicated multi-stepped shape according to the complexity of the chemical reaction.

In some embodiments, and depending on the type of voltammetry used, the potential is varied continuously or stepwise or in pulses.

Exemplary potentials that can be applied to a sensing electrode as described herein typically range from 0 to about −2 Volts.

Voltammetry experiments can be categorized as linear sweep voltammetry and cyclic voltammetry.

Cyclic voltammetry is the process of electrochemical analysis in which the applied voltage is of a multi or mono-triangular shape. The resulting plot of current versus linear triangular potential scan of the working electrode is called cyclic voltammogram, while the plot of current versus linear potential scan of the working electrode is called linear sweep voltammogram. Cyclic voltammetry is usually the preliminary process used to determine the reduction potential of an analyte, the media's influence and the thermodynamics, as well as kinetics, of the electrochemical reaction.

In response to the triangular shaped potential, the measured current of the electrochemical cell that contained initially only the oxidized species, gradually increases up to a sharp peak at E_{p[red]}, followed by current decrease when most species adjacent to the electrode surface are reduced. When reversing the potential's direction, a gradual increase of current at the opposite direction ends in a sharp peak at E_{p[ox]}, where the chemical reaction proceeds to the opposite direction towards the oxidized form. When most species adjacent to the electrode surface are oxidized, the current decreases until the point of potential reverses, and so on.

Since an electrochemical reaction is located at the interface between the working electrode and the electrolyte solution, the reduced and oxidized species causing the sharp peaks of the voltammogram are concentrated to a narrow diffusive layer adjacent to the electrode. As a result, the shape of the curve's peak depends on the rate of diffusion. The peak's incline correlative to the concentration of electroactive particles on the electrode's surface, while the sharp decline depends solely on time, and results from the absence of electroactive particles near the surface due to limited diffusion.

In order to increase the sensitivity of voltammetric measurements, the share of the Faradic currents in the obtained voltammogram can be increased on the expense of the nonfaradaic background current. Such alterations are enabled by applying a series of short duration potential steps (each last for several milliseconds) in a technique termed “pulse voltammetry”. At the end of each potential step, two different current decay rates are obtained: sharp exponential decay to a negligible level is characteristic to the charging current, while slower decay is typical to the Faradic current. By recording the current's signal at the later regime, more of the signal is attributed to the Faradic current, while the contribution of the charging current is negligible. The differential pulse voltammogram is obtained from the subtraction of the pre-pulse current from the current that is obtained after the pulse is switched off, plotted against the applied potential. The corresponding sensitivity is thereby increased. The differential pulse voltammetry techniques vary by the shape of the applied potential waveform, and the current sampling technique.

Alongside increased sensitivity, differential pulse voltammetry allow the detection of two different analytes with similar redox potentials, by analysis of the peak's width according to the number of electrons that participate in their redox reaction. Exemplary values used for differential voltammetry measurements are 25-50 mV for current pulse amplitudes and 5 mV/second for the scan rate, while steeper amplitudes and faster scan rates are also contemplated.

In some of any of the embodiments described herein, an electrochemical parameter measured in a method as described herein is a change in electrical current relative to a derivative of the applied potential, although any other voltammogram is contemplated.

In some of any of the embodiments described herein, the measured electrochemical parameter is processed by a signal processor, as described herein in any of the respective embodiments, to thereby determine a presence and/or a level (amount) of the viral biomarker to be detected, in the sample.

In some of any of the embodiments described herein, the method further comprises, prior to contacting the sensing electrode with agent 122 or a solution containing same (e.g., electrolyte), measuring an electrochemical parameter as described herein of electrode 100 when contacted with agent 122 or a solution containing same, or measuring an electrochemical parameter as described herein of electrode 120 that does not contain a viral biomarker. For each of the above options, the measurement of the electrochemical parameter measures a background or control signal, which is provided by an electrode that does not have the viral biomarker adsorbed thereto. In some embodiments, upon measuring the electrochemical parameter resulting from contacting sensing electrode 120 and the sample, the background signal is subtracted from the measured electrochemical parameter.

According to some of any of the embodiments described herein, the electrolyte (e.g., electrolyte 18) further comprises an electroactive agent (e.g., agent 124), which is also referred to herein as electrochemically reactive substance or agent, that undergoes an electrochemically detectable (e.g., redox) reaction in response to an interaction between the viral biomarker in electrode 120 and agent 122, to thereby generate a change in an electrochemical parameter.

According to some of any of the embodiments described herein, the biomarker, the substance that interacts therewith (e.g., agent 122) and the electroactive agent (e.g., agent 124) are selected such that an interaction between the biomarker and the substance generates a moiety or species, and said the electroactive agent (e.g., agent 124) undergoes an electrochemically detectable (e.g., redox) reaction in response to a presence of the chemical moiety or species.

In exemplary embodiments, and as mentioned hereinabove, the chemical moiety or species comprises a proton. In some of these embodiments, the electroactive agent (e.g., agent 124) is a pH-dependent redox reactive agent, that undergoes a pH-dependent electrochemically detectable (e.g., redox) reaction. In some embodiments, the interaction between the viral biomarker and the substance results in a pH change, which is electrochemically detectable by the electroactive agent (e.g., agent 124).

In some of any of the embodiments described herein, an electrochemical cell or a sensing system comprising same as described herein (e.g., cell 10) is operable by assembling at least a sensing electrode as described herein and an electrolyte containing at least agent 122 and preferably also agent 124 as described herein, and electric means for electrically connecting the sensing electrode to a power source; contacting sensing electrode with the electrolyte solution containing agent 122; applying a potential to the sensing electrode, by means of a power source as described herein; and measuring an electrochemical signal that is indicative of an electrochemically-detectable reaction in which agent 122 participates. In some embodiments, the electrochemical signal is an electrical current generated at the sensing electrode is response to said potential, and measuring the signal is effected by means of an electrical current measuring device. The measured current is indicative of a presence and/or level (e.g., amount, concentration) of the viral biomarker in electrode 120, which is also indicative of a presence and/or level of a viral infection in subject in case the sample is drawn from the subject.

In some embodiments, the electrochemical cell comprises a reference electrode and applying a potential is effected by applying voltage between the sensing electrode and the reference electrode.

The power source is configured to apply potential to the sensing electrode according to any known voltammetry method, as described in further detail hereinafter, in embodiments related to a sensing method.

In some embodiments, the power source is configured to apply a varying potential to the sensing electrode, as described herein in any of the respective embodiments.

In some embodiments, the system or electrochemical cell is configured to determine a current generated in response to the varying potential, and in some embodiments, the system or electrochemical cell is configured for determining a change in the current generated at the sensing electrode, in response to the varying potential.

Generally, but not necessarily, the system or electrochemical cell is configured for providing a voltammogram that presents values that are in line with the voltammetry methodology used.

Determination of a change in the electrical current, according to any of the respective embodiments, can be performed by means of a device which is configured to process the received signals (e.g., the mode of the applied varying potential and corresponding generated current data) so as to provide a value or a set of values as desired (e.g., a change in electrical current relative to a derivative of the applied potential, or any other voltammogram). Such a device is also referred to herein as a signal processor.

In some embodiments, the signal processor is a data processor such as a computer configured for receiving and analyzing the signals. The signal processor extracts, from each generated signal or set of signals, a parameter (e.g., a voltammogram) that is indicative of the electrochemical reaction, and hence of a presence and/or level of the viral biomarker and accordingly the presence and/or level of a viral infection if desired, as described herein.

In some embodiments of the invention the signal processor is configured to construct a fingerprint of the viral biomarker, for example, a voltammogram obtained upon contacting an electrolyte 18 containing agent 122 and optionally agent 124 with electrode 120 and applying a certain mode of a varying potential (e.g., a differential pulse potential).

In some of any of the embodiments of the invention the signal processor is configured to determine a level of a viral biomarker in electrode 120, by accessing and/or processing relevant data. Such data can include, for example, a calibration curve, e.g., of voltammograms, or of specific values obtained in voltammetry measurements (e.g., a reduction peak), obtained for varying concentrations of the viral biomarker, and stored on a computer readable medium. For example, the signal processor may access the calibration curve, search for a value (e.g., a concentration) that matches the value obtained upon operating the system, and identify a concentration of the viral biomarker that matches this value. Alternatively, or in addition, the data include a lookup table stored on a computer readable medium, which can be searched for values that match the measured value and are indicative of a level of the viral biomarker. Further alternatively, or in addition, the data include a predetermined relationship between the measured value and a level of the viral biomarker. For example, if such a predetermined relationship comprises a linear relationship, the signal processor can determine the level of the viral biomarker by means of extrapolation, based on the pre-determined relationship.

Once the presence and/or amount of the viral biomarker in a sample is obtained, it can be transmitted to a remote location. Also contemplated are embodiments in which the electric signal produced by the reaction is transmitted to a remote location at which it can be analyzed to determine the amount of the viral biomarker. The electric signal can be transmitted as a raw signal or it can be processed prior to the transmission. For example, in some embodiments, the signal is digitized prior to sending to provide a digital signal, wherein the transmitted signal is the digital signal.

### Uses:

The electrode, method and system as described herein in any of the respective embodiments are usable in determining a presence and/or amount of a viral infection in a biological sample, or simply in determining a presence and/or amount of a viral biomarker (e.g., for research purposes).

A sample as described herein can be a biological sample.

Exemplary biological samples include, but are not limited to, blood (e.g., peripheral blood leukocytes, peripheral blood mononuclear cells, whole blood, cord blood), saliva, a solid tissue biopsy, cerebrospinal fluid, urine, lymph fluids, and various external secretions of the respiratory, intestinal and genitourinary tracts, synovial fluid, amniotic fluid and chorionic villi.

Biopsies include, but are not limited to, surgical biopsies including incisional or excisional biopsy, fine needle aspirates and the like, complete resections or body fluids. Methods of biopsy retrieval are well known in the art.

In some embodiments, the biological sample is of subject suspected as having the viral infection associated with the viral biomarker.

According to preferred embodiments, the biological sample is a saliva sample of the subject. The saliva sample can be drawn from the subject and then be contacted with an electrode or an electrochemical system as described herein, or, the electrode (e.g., electrode 102) can be configured so as to contact a subject's saliva (e.g., by contacting an oral cavity of the subject) as is shown, for example, in FIG. 1C, and thereafter, the thus obtained electrode (e.g., electrode 120) is integrated with an electrochemical system as described herein.

According to some of any of the embodiments described herein, a pH of the saliva of the subject is in a range of from 6 to 8.

According to some of any of the embodiments described herein, a time period between contacting the biological sample with the electrode and operating an electrochemical cell that comprises the electrode is up to 10 hours, or up to 1 hour, or up to 30 minutes, or up to 10 minutes.

According to some of any of the embodiments described herein, the change in the electrochemical parameter is generated within a time period of up to 5 minutes, for example, between 30 seconds and 5 minutes, or between 1 minute to 3 minutes, from operating an electrochemical cell that comprises the electrode.

According to some of any of the embodiments described herein, a concentration of the biomarker in the sample can be lower than 100 micrograms per ml sample.

According to some of any of the embodiments described herein, the biomarker is SARS-CoC-2 3CL^{pro}, and the method is of determining a presence and/or amount of a viral infection caused by SARS-CoV-2 in the subject.

According to an aspect of some embodiments of the present invention, there is provided a method of determining a presence of a viral infection associated with 3CL^{pro} in a subject, the method comprising contacting a saliva sample of the subject with a probe selective to the 3CL^{pro}, the probe being such that generates a detectable signal in response to a presence of 3CL^{pro} in the sample.

According to exemplary embodiments, the probe in an electrode as described herein in any of the respective embodiments, and the electrode is used as described herein.

According to some of any of the embodiments described herein, the method is capable of a quantification of a virus in a sample. In some embodiments, the quantification is determined for the virus in a concentration in a range of from 0.1 μg ml⁻¹ to 10 mg ml⁻¹, or from 1 μg ml⁻¹ to 1 mg ml⁻¹, or from 5 μg ml⁻¹ to 1 mg ml⁻¹, or from 5 μg ml⁻¹ to 100 μg ml⁻¹, or from 5 μg ml⁻¹ to 500 μg ml⁻¹, or from 5 μg ml⁻¹ to 500 μg ml⁻¹, or from 10 μg ml⁻¹ to 200 μg ml⁻¹.

According to some of any of the embodiments described herein, the biological sample is a saliva sample and the contacting is effected by contacting the electrode with the oral cavity of the subject.

According to an aspect of some embodiments of the present invention there is provided a method of determining a presence of a viral infection caused by SARS-CoV-2 in a subject, the method comprising determining a presence of 3CL^{pro} as described herein in a saliva sample of the subject.

### Kits:

According to the present embodiments, there are provided kits that are usable in the methods as described herein.

A kit, according to some of the present embodiments, can comprise electrode 100 as described herein, and a sensing agent as described herein, packaged individually within the kit. The kit may further comprise an agent that interferes with an interaction of biological species or materials with the electrode, as described herein.

Electrode 100 can be a pre-treated electrode, as described herein, for example, laminated, as described herein. An exemplary electrode is shown in FIG. 1A.

The sensing agent, the additional agent, if present, and optionally washing solution, can all be included in the kit, preferably packaged individually.

Alternatively, one or more of the above is not included in the kit, and the kit may comprise instructions to treat the electrode packaged therein with the sensing agent or one or more of the additional components.

Further alternatively, the kit may comprise electrode 102, that is, an electrode having attached thereto the sensing agent, as described herein.

According to some embodiments, the kit may further comprise agent 122 as described herein, optionally in a solution, for example, in electrolyte solution 18 as described herein, preferably individually packaged in the kit. The kit may comprise instructions to use or prepare electrode 102, contact it with a sample as described herein, and then contact the electrode with agent 122 or a solution containing same as described herein. The may further comprise instructions to integrate the electrode, upon contacting the sample, with an electrochemical cell or system as described herein, while using agent 122 or a solution comprising same.

According to some embodiments, the kit may further comprise agent 122 as described herein, optionally in a solution, for example, in electrolyte solution 18 as described herein, preferably individually packaged in the kit. The kit may comprise instructions to use or prepare electrode 102, contact it with a sample as described herein, and then contact the electrode with agent 122 or a solution containing same as described herein. The kit may further comprise instructions to integrate the electrode, upon contacting the sample (e.g., electrode 120), with an electrochemical cell or system as described herein, while using agent 122 or a solution comprising same.

According to some embodiments, the kit may further comprise an electrolyte solution (e.g., electrolyte 18), either per se, or containing agent 122 and/or agent 124 as described herein, preferably individually packaged in the kit.

According to some embodiments, the kit may further comprise agent 124 as described herein, optionally in a solution, for example, in electrolyte solution 18 as described herein, preferably individually packaged in the kit. The kit may comprise instructions to use or prepare electrode 102, contact it with a sample as described herein, and then contact the electrode with a solution containing agent 122 and agent 124 as described herein. The kit may further comprise instructions to integrate the electrode, upon contacting the sample (e.g., electrode 120), with an electrochemical cell or system as described herein, while using agents 122 and 124 or a solution comprising same.

In some embodiments, the kit may further comprises electrochemical cell 10, or components thereof, to be assembled with electrode 120 for conducting the electrochemical measurements. The electrochemical cell can comprise means for connecting it to a power source and/or a portable power source such as a battery.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Experimental Methods

### Materials:

CPE (254 μm thick, type Spectracarb 2050A-1050, Engineered Fibers Technology), Ag/AgCl reference electrode (type RE-1CP, ALS ltd.), Pt electrode (99.999%, 1 mm diameter, Holland-Moran ltd.), 3CL^{pro} enzyme (Recombinant derived from Escherichia coli, ab277614, ABCAM; SEQ ID NO:2), 3CL^{pro} specific antibody (Rabbit-derived, NBP3-07062, Novus Biologicals®; binding to SEQ ID NO:3), 3CL^{pro} substrate (KTSAVLQSGFRKME, Sigma-Aldrich™; SEQ ID NO:1), GFP (Recombinant derived from Escherichia coli, ab84191, ABCAM; SEQ ID NO:6), myoglobin antibody (Monoclonal rabbit-derived, ab77232, ABCAM; binding to SEQ ID NO:5), HIV-2 Protease (Recombinant derived from Escherichia coli, ab84117, ABCAM; SEQ ID NO:10), CA-15.3 (Recombinant derived from Escherichia coli, ab80082, ABCAM: SEQ ID NO:11), Human TMPRSS2 protein (Recombinant derived from Wheat germ, ab112364, ABCAM; SEQ ID NO: 7), MERS-CoV 3CL Protease (Recombinant derived from Escherichia coli, E-719, Novus Biologicals®; SEQ ID NO:8), SARS-CoV 3CL Protease (Recombinant derived from Escherichia coli, E-718, Novus Biologicals®; SEQ ID NO:9), Chymotrypsin protein (Native human, ab90927, ABCAM; SEQ ID NO: 12), bovine serum albumin (BSA) was obtained from Sigma Aldrich™ (SEQ ID NO:4), p-benzoquinone (reagent grade, Sigma-Aldrich™), Acetone (9005-68, J. T. Baker), Isopropanol (IPA, 9079-05, J. T. Baker), Deionized water (DW, 18 MΩ·cm), PBS (40 mm NaCl, 10 mm phosphate buffer, and 3 mM KCl, pH 7.4; Sigma-Aldrich™), Disodium hydrogen phosphate (S7907, Sigma-Aldrich™), GenSure™ COVID-19 Antigen Rapid Test Kit (P2004s, GenSure®).

### Electrode Fabrication and Immuno-Functionalization:

Carbon paper was cut into rectangular pieces of 7×50 mm, laminated with polyethylene at 75° C. to prevent solution capillary rising and contact wetting. An active window of 4 mm diameter was designed and left un-laminated out of the CPE.

For immuno-functionalization, CPE was washed with IPA and distilled water, then 2 μl of 3CL^{pro} antibody were drop-casted on CPE's active window. CPE was then washed well with PBS, optionally soaked for 20 minutes in BSA (5 mg ml⁻¹), and washed again with PBS.

### Measurements of Enzymatic pH Change:

Fluorescence of HPTS (Excitation 430 nm, emission 470 nm) of 80 μl of HPTS (80 pmol), 3CL^{pro} (80 pmol), and 3CL^{pro} substrate (8 nmol) in 10 mM disodium hydrogen phosphate pH 7.5 was measured at different times. pH values were calculated out of a calibration curve, shown in FIG. 2E. Control was 80 μl of HPTS (80 pmol) and 3CL^{pro} (80 pmol) in 10 mm disodium hydrogen phosphate pH 7.5.

### Electrochemical Measurements:

All electrochemical experiments were performed using a potentiostat (EmStat³, PalmSense) using PSTrace 5.6 Software. CV measured from −0.3 V to 0.5 V, scan rate 0.1 V sec⁻¹. A 3-electrode cell was used, with commercial Ag/AgCl as reference electrode and Pt as a counter electrode. Measurements were conducted in 900 μl of 25 mM PB, 75 mM NaCl, 32 μM 3CL^{pro} substrate, and 15 μM p-benzoquinone (pBQ).

### Imaging:

Light microscopy used Olympus BX41m-LED with the use of a U-PMTCV camera adapter in dark-field mode. Scanning electron microscopy (SEM) imaging used Quanta 200FEG ESEM, Thermo Scientific™, 20.0 kV, WD 10.0 mm, and high-resolution SEM (HR-SEM) imaging used GemeniSEM-300, Zeiss, 0.500 kV, WD 4.6 mm.

### Protein Adsorption, Desorption, and Binding Measurements:

Proteins were quantified using NanoDrop One/OneC spectrophotometer, Thermo Scientific. For adsorption, CPE was soaked in protein solutions, and the adsorbed amounts at a time point tᵢ were calculated using equation (1):

$$Adsorption ( {t}_{i} ) = \frac{{C}_{0} - {C}_{i}}{{C}_{0}} \times 100 \% \qquad ( 1 )$$

Where C₀ is the initial concentration and Cᵢ is the concentration at tᵢ. Binding and desorption were calculated in the same fashion.

### Atomic Percentage Measurements:

X-Ray Photoelectron Spectroscopy (XPS) was measured under ultra-high vacuum (UHV; 2.5×10⁻¹⁰ Torr base pressure) using Thermo Scientific™ Nexsa G2 System.

Atomic percentage was calculated using Thermo Scientific™ Avatage software using the following equation:

$${C}_{A} = \frac{{I}_{A} / {S}_{A}}{{\sum }_{n} {I}_{n} / {S}_{n}} \times 100 \%$$

Where C_{A} is the atomic % content of A, I_{A} is the intensity of an atom's peak and S_{A} is the sensitivity of the atom.

### Clinical Sample Preparation and Testing:

Approval for the human saliva experiments was received from the Ethics Committee of Tel Aviv University. Saliva samples were collected in sterile 15 ml tubes and kept refrigerated until measuring. No pretreatment steps were taken before measurements.

### Sequences:

Table 1 below presents the amino acid sequences of the peptide and proteins used in these studies.

TABLE 1

| Construct | | | Mw | SEQ |
|---|---|---|---|---|
| name | Composition | Sequence | (kDa) | ID NO. |
| SARS-CoV-2 | Peptide | KTSA VLQS GFRK ME | 1.6 | 1 |
| 3CL^{pro} | | | | |
| Substrate | | | | |
| SARS-CoV-2 | E. coli- | SGFR KMAF PSGK VEGC | 34 | 2 |
| 3CL^{pro} | Derived | MVQV TCGT TTLN GLWL | | |
| | SARS-COV-2 | DDVV YCPR HVIC TSED MLNP | | |
| | 3CL^{pro} | NYED LLIR KSNH NFLV QAGN | | |
| | protein | VQLR VIGH SMQN CVLK | | |
| | | LKVD TANP KTPK YKFV RIQP | | |
| | | GQTF SVLA CYNG SPSG VYQC | | |
| | | AMRP NFTI KGSF LNGS CGSV | | |
| | | GFNI DYDC VSFC YMHH MELP | | |
| | | TGVH AGTD LEGN FYGP | | |
| | | FVDR QTAQ AAGT DTTI TVNV | | |
| | | LAWL YAAV INGD RWFL | | |
| | | NRFT TTLN DFNL VAMK | | |
| | | YNYE PLTQ DHVD ILGP LSAQ | | |
| | | TGIA VLDM CASL KELL | | |
| | | QNGM NGRT ILGS ALLE DEFT | | |
| | | PFDV VRQC SGVT FQ | | |
| SARS-CoV-2 | E. coli- | SMQN CVLK LKVD TANP | | 3 |
| 3CL^{pro} | Derived | KTPK YKFV RIQP GQTF SVLA | | |
| antibody | SARS-COV-2 | CYNG SPSG VYQC AM | | |
| binding | 3CL^{pro} | | | |
| domain | protein | | | |
| (epitope) | | | | |
| BSA | Native | MKWV TFIS LLLL FSSA YSRG | 69 | 4 |
| | Bovine | VFRR DTHK SEIA HRFK DLGE | | |
| | Serum | EHFK GLVL IAFS QYLQ QCPF | | |
| | Albumin | DEHV KLVN ELTE FAKT | | |
| | (BSA) protein | CVAD ESHA GCEK SLHT LFGD | | |
| | | ELCK VASL RETY GDMA | | |
| | | DCCE KQEP ERNE CFLS HKDD | | |
| | | SPDL PKLK PDPN TLCD EFKA | | |
| | | DEKK FWGK YLYE IARR HPYF | | |
| | | YAPE LLYY ANKY NGVF | | |
| | | QECC QAED KGAC LLPK IETM | | |
| | | REKV LASS ARQR LRCA SIQK | | |
| | | FGER ALKA WSVA RLSQ KFPK | | |
| | | AEFV EVTK LVTD LTKV HKEC | | |
| | | CHGD LLEC ADDR ADLA KYIC | | |
| | | DNQD TISS KLKE CCDK PLLE | | |
| | | KSHC IAEV EKDA IPEN LPPL | | |
| | | TADF AEDK DVCK NYQE | | |
| | | AKDA FLGS FLYE YSRR HPEY | | |
| | | AVSV LLRL AKEY EATL EECC | | |
| | | AKDD PHAC YSTV FDKL | | |
| | | KHLV DEPQ NLIK QNCD QFEK | | |
| | | LGEY GFQN ALIV RYTR KVPQ | | |
| | | VSTP TLVE VSRS LGKV GTRC | | |
| | | CTKP ESER MPCT EDYL SLIL | | |
| | | NRLC VLHE KTPV SEKV TKCC | | |
| | | TESL VNRR PCFS ALTP DETY | | |
| | | VPKA FDEK LFTF HADI CTLP | | |
| | | DTEK QIKK QTAL VELL KHKP | | |
| | | KATE EQLK TVME NFVA | | |
| | | FVDK CCAA DDKE ACFA | | |
| | | VEGP KLVV STQT ALA | | |
| Myoglobin | | GHHE AEIK PLAQ SHAT KHKI | ~150 | 5 |
| antigen | | PVKY LEFI SECI IQVL QSKH | | |
| portion to | | PGDF GADA QGAM NKAL | | |
| which an | | ELFR KDMA SNYK ELGF QG | | |
| exemplary | | | | |
| rabbit-derived | | | | |
| antibody | | | | |
| binds | | | | |
| GFP | Escherichia | MSKG EELF TGVV PILV ELDG | 27 | 6 |
| | coli-derived | DVNG HKFS VSGE GEGD | | |
| | | ATYG KLTL KFIC TTGK LPVP | | |
| | | WPTL VTTF SYGV QCFS RYPD | | |
| | | HMKQ HDFF KSAM PEGY | | |
| | | VQER TIFF KDDG NYKT RAEV | | |
| | | KFEG DTLV NRIE LKGI DFKE | | |
| | | DGNI LGHK LEYN YNSH NVYI | | |
| | | MADK QKNG IKVN FKIR HNIE | | |
| | | DGSV QLAD HYQQ NTPI GDGP | | |
| | | VLLP DNHY LSTQ SALS KDPN | | |
| | | EKRD HMVL LEFV TAAG ITHG | | |
| | | MDEL YK | | |
| Human | Wheat germ- | GWGA TEEK GKTS EVLN | 38 | 7 |
| TMPRSS2 | derived | AAKV LLIE TQRC NSRY VYDN | | |
| | | LITP AMIC AGFL QGNV DSCQ | | |
| | | GDSG GPLV TSKN NIWW LIGD | | |
| | | TSWG SGCA KAYR PGVY | | |
| | | GNVM VFTD WIYR QMRA DG | | |
| MERS-CoV | Escherichia | SGLV KMSH PSGD VEAC | 34 | 8 |
| 3CL^{pro} | coli-derived | MVQV TCGS MTLN GLWL | | |
| | | DNTV WCPR HVMC PADQ | | |
| | | LSDP NYDA LLIS MTNH SFSV | | |
| | | QKHI GAPA NLRV VGHA | | |
| | | MQGT LLKL TVDV ANPS | | |
| | | TPAY TFTT VKPG AAFS VLAC | | |
| | | YNGR PTGT FTVV MRPN YTIK | | |
| | | GSFL CGSC GSVG YTKE GSVI | | |
| | | NFCY MHQM ELAN GTHT | | |
| | | GSAF DGTM YGAF MDKQ | | |
| | | VHQV QLTD KYCS VNVV | | |
| | | AWLY AAIL NGCA WFVK | | |
| | | PNRT SVVS FNEW ALAN QFTE | | |
| | | FVGT QSVD MLAV KTGV | | |
| | | AIEQ LLYA IQQL YTGF QGKQ | | |
| | | ILGS TMLE DEFT PEDV NMQI | | |
| | | MGVV MQ | | |
| SARS-CoV | Escherichia | SGFR KMAF PSGK VEGC | 34 | 9 |
| 3CL^{pro} | coli-derived | MVQV TCGT TTLN GLWL | | |
| | | DDTV YCPR HVIC TAED MLNP | | |
| | | NYED LLIR KSNH SFLV QAGN | | |
| | | VQLR VIGH SMQN CLLR | | |
| | | LKVD TSNP KTPK YKFV RIQP | | |
| | | GQTF SVLA CYNG SPSG VYQC | | |
| | | AMRP NHTI KGSF LNGS CGSV | | |
| | | GFNI DYDC VSFC YMHH MELP | | |
| | | TGVH AGTD LEGK FYGP | | |
| | | FVDR QTAQ AAGT DTTI TLNV | | |
| | | LAWL YAAV INGD RWFL | | |
| | | NRFT TTLN DENL VAMK | | |
| | | YNYE PLTQ DHVD ILGP LSAQ | | |
| | | TGIA VLDM CAAL KELL | | |
| | | QNGM NGRT ILGS TILE DEFT | | |
| | | PFDV VRQC SGVT FQ | | |
| HIV-2 | Escherichia | PQFS LWKR PVVT AHIE GQPV | 11 | 10 |
| Protease | coli-derived | EVLL DTGA DSI VAGI ELGS | | |
| | | NYSP KIVG GIGG FINT KEYK | | |
| | | NVEI EVLN KRVR ATIM TGDT | | |
| | | PINI FGRN ILAS GMS LNL | | |
| CA-15.3 | Escherichia | LRPG SVVV QLTL AFRE GTIN | 14 | 11 |
| | coli-derived | VHDV ETQF NQYK TEAA | | |
| | | SRYN LTIS DVSG | | |
| Chymotrypsin | Native human | MLGITVLAALLACASSC | 25 | 12 |
| | Chymotrypsin | GVPSFPPNLSARVVGGE | | |
| | protein | DARPHSWPWQISLQYL | | |
| | | KNDTWRHTCGGTLIAS | | |
| | | NFVLTAAHCISNTRTYR | | |
| | | VAVGKNNLEVEDEEGS | | |
| | | LFVGVDTIHVHKRWNA | | |
| | | LLLRNDIALIKLAEHVE | | |
| | | LSDTIQVACLPEKDSLL | | |
| | | PKDYPCYVTGWGRLW | | |
| | | TNGPIADKLQQGLQPV | | |
| | | VDHATCSRIDWWGFR | | |
| | | VKKTMVCAGGDGVIS | | |
| | | ACNGDSGGPLNCQLEN | | |
| | | GSWEVFGIVSFGSRRGC | | |
| | | NTRKKPVVYTRVSAYID | | |
| | | WINEKMQL | | |

### Example 1

### Sensor Design and Mode of Action

The CPE is fabricated from a conductive carbon paper that contains multi-layers of micro-carbon-fibers (μCF) as a 3D matrix with an ultra-high surface area of 1000-2500 m² gram⁻¹ [Krivitsky et al., ACS Sens. 2021, 6, 1187; Krivitsky et al., Anal. Chem. 2019, 91, 5323; and Williams et al., Appl. Environ. Microbiol. 2001, 67, 2453]. Carbon is an attractive material for electrochemical-based sensor development, owing to the well-known chemistry [J. Wang, Analytical Electrochemistry: Wang/Analytical Electrochemistry, Third Edition, John Wiley & Sons, Inc., Hoboken, NJ, USA, 2006], high conductivity, relatively low background currents, and high analytical signal [Sun et al., Materials Today 2015, 18, 215; and R. Mohammadzadeh Kakhki, Arabian Journal of Chemistry 2019, 12, 1783]. One cm² CPE weighs only about 12 milligram (mg), therefore the CPE's detection window of 0.13 cm² displays a ca. 3.90 m² of active working electrode area.

The design of an exemplary electrode and scanning electron microscopy (SEM) images of the μCF are shown in FIG. 1A. As illustrated in FIG. 1B, 3CL^{pro} is targeted specifically by implementing a surface-embedded specific antibody. The 3CL^{pro}-specific antibody was drop-casted and physically adsorbed onto the CPE surface. The modification process relies on a single antibody, and requires two soaking steps with no covalent modification steps required.

Once 3CL^{pro} binds to the embedded antibody, it interacts with its substrate (SEQ ID NO: 1; which is presented in the electrolyte) by hydrolyzing it, and this interaction generates protons, and changes the electrolyte pH. A RedOx reactive pH indicator is used to electrochemically detect the pH change brought by the substrate's (SEQ ID NO: 1) surface-bound 3CL^{pro} enzymatic hydrolysis (to generate SEQ ID NOs: 14 and 15), as schematically illustrated in FIG. 1D. The diagnostic signal is amplified by relying on the enzymatic activity turnover rate. Each protease molecule performs hydrolysis of about 60 substrate molecules per minute [Kao et al., FEBS Letters 2004, 576, 325], resulting in signal amplification of at least 120-fold within 2 minutes. A library of substrates has been recognized for 3CL^{pro} (see, for example, SEQ ID NOs: 1 and 16-36); the 3CL^{pro} substrate used in the Examples herein (SEQ ID NO: 1) showed a high affinity and turnover rate [Chan et al., Discovery of SARS-CoV-2 M^{pro} Peptide Inhibitors from Modelling Substrate and Ligand Binding, Chem. Sci. 2021, 12, 13686-13703; Grum-Tokars et al., Virus Res 2008, 133, 63; El-Baba et al., Angewandte Chemie International Edition 2020, 59, 23544; and Hoffman et al., J. Med. Chem. 2020, 63, 12725].

### Example 2

### 3CL^{pro} Activity and RedOx pH Indicator Characterization

To quantify the pH change brought by the proteolytic activity of 3CL^{pro}, 8-Hydroxypyrene-1,3,6-trisulfonic acid (HPTS) was used as a fluorogenic pH indicator [Willoughby et al., Pflugers Arch. 1998, 436 (4), 615-622]. The pH measurements of 80 pmol 3CL^{pro} (SEQ ID NO: 2) activity in the presence of 8 nmol 3CL^{pro} substrate (SEQ ID NO: 1) are shown in FIG. 2A. The pH drops 0.63 units within two minutes since the theoretical pKa of the substrate peptide fragment is about 2.5; this indicates that about 90 pmol of the substrate was enzymatically cleaved. For a cell volume of 900 μl, the same amount of 3CL^{pro} would theoretically yield a pH change of about 0.74 units, as follows:

Measurements of 3CL^{pro} (SEQ ID NO: 2) activity showed 80 pmol 3CL^{pro} activity in the presence of 8 nmol 3CL^{pro} substrate (SEQ ID NO: 1) results in a pH drop of 0.63 units (starting pH was 7.51 and final pH was 6.88) in 2 minutes, see FIG. 2A. Since 3CL^{pro} tends to dimerize, and the turnover rate of the tested 3CL^{pro} substrate is about 60 substrate molecules per minute, the activity of 3CL^{pro} could be calculated by predicting that 80 pmol 3CL^{pro} would cleave 1.2-2.4 nmol of the substrate in 120 seconds, for 80 μl well:

$$KTSAVLQSGFRKME ( SEQID NO : 1 ) {\to }^{3 C {L}^{p r o}} KTSAVLQ ( COOH ) + ( {H}_{2} N ) SGFRKME ( [ SEQ ID NO : 14 ] ( COOH ) + ( H 2 N ) [ SEQID NO : 15 ] )$$

$$KTSAVLQ ( COOH ) ⇌ KTSAVLQ ( {COO}^{-} ) + {H}^{+} \frac{d [ {H}^{+} ]}{dt} = k [ KTSAVLQ ( COOH ) ]$$

$$\to \frac{0.101 μM}{120 sec} = k \times ( 1.5 - 3. μM ) \to$$

$$k = ( 2.8 - 5.6 ) \times {10}^{- 4} \frac{1}{sec}$$

For 900 μl cell with the same presence of 3CL^{pro}, and starting pH is 7.40:

$$\frac{d [ {H}^{+} ]}{d t} = ( 2.8 - 5 \text{.6} ) \times {10}^{- 4} \frac{1}{sec} \times ( 0.13 - 0.27 μM ) \to d [ {H}^{+} ] = 0.05 - 0.18 μM$$

$${[ {H}^{+} ]}_{f} = 0.09 - 0.22 μM$$

$$\to pH = 6.66 - 7.05 \to ΔpH = 0.35 - 0.74$$

The pH plateaus at 8 minutes, even though there is excess substrate available, is to be noted. It has been reported that 3CL^{pro} goes through 3D structure changes with pH changes in the window from 7.6 to 6.0 [Chou et al., Biochemistry 2004, 43, 14958], possibly affecting enzymatic efficiency, which is reported to be maximal around pH 7 [Fan et al., J Biol Chem 2004, 279, 1637]. Therefore, 3CL^{pro} activity is self-limiting, with pH being an in-vitro stop-signal, and it can be inferred that there is no necessity for incubation time longer than two minutes under these conditions.

In order to translate the enzymatic reaction to an electrochemically-detectable parameter, the pH change as a result of the enzymatic reaction was exploited, using an electroactive agent that participates in an electrochemical reaction in response to pH change. Such an agent is also referred to herein as a redox reaction pH indicator or as a pH-dependent redox probe.

A RedOx reactive pH indicator able to indicate the expected pH change in the active enzymatic range was therefore used. In this context, several quinones have been shown to change their electrochemical RedOx potential under different pH environments [Bailey and Ritchie, Electrochimica Acta 1985, 30, 3; Cobb et al., J. Am. Chem. Soc. 2019,141, 1035]. p-Benzoquinone (pBQ) was chosen as an exemplary pH-dependent RedOx probe. pBQ undergoes a two-electron reduction reaction, accompanied by a reaction with up to two protons (2e⁻/2H⁺), depending on the solution pH, as shown in FIG. 2B. The RedOx peaks shift (at pH lower than 10) is derived from equation (2) [Bailey and Ritchie, 1985, supra; Cobb et al., 2019, supra; Wang et al., J. Electrochem. Soc. 2015, 163, H201; and Quan et al., J. Am. Chem. Soc. 2007, 129, 12847]:

$${E}_{p H}^{0} = {E}_{p H 7}^{0} - \frac{2.3 R T}{nF} m \times pH \qquad ( 2 )$$

Where E⁰ is the reaction standard potential, R is the universal gas constant, T is the temperature in Kelvin, n is the number of electrons transferred, F is the Faraday constant, and m is the number of protons transferred. When n=2, m=2, and T=298K, the potential change expected per pH unit is [Lahav et al., Electroanalysis 1998, 10, 1159]:

$$\frac{\partial {E}_{p H}^{0}}{\partial pH} = \frac{2.3 R T}{F} \approx 60 \frac{mV}{pH} \qquad ( 3 )$$

The peak shifts of solutions comprising pBQ are evident in FIG. 2C and linearly plotted in FIG. 2D. CV measurements were highly repetitive, as demonstrated in FIG. 5.

While both plots in FIG. 2D fit well with the linear trend (R²>0.91), oxidation peak shifts showed better fittings and more significant peak potential shifts. Consequently, the oxidation peak shift was chosen as the detection marker. The peak shift as a response of 3CL^{pro} activity has been calculated using equation (4):

$$Δ {E}_{P e a k} = {E}_{P e a k}^{Substrate} - {E}_{P e a k}^{Sample} \qquad ( 4 )$$

Where E_{Peak}^{sample} is the voltage at maximal oxidation current after 2 minutes of CPE incubation in the sample, and E_{Peak}^{Substrate} is the voltage at maximal oxidation current after adding 3CL^{pro} substrate.

### Example 3

### Immuno-Functionalization of the Carbon Electrode

Protein permeation through the CPE's μCF matrix was tested by confocal fluorescence microscopy of untreated (bare) CPE and was compared with GFP (SEQ ID NO: 6)-treated CPE. The images and fluorescence intensity curves are presented in FIG. 6A-C, and show protein permeating through the full depth of the μCF matrix. Proteins quickly and strongly adsorb to the CPE surface through electrostatic attraction, as evident in the antibody adsorption plot, shown in FIG. 3A. The high surface area of the CPE allows for a very high antibody density per geometric area compared to a planar surface. The results in FIG. 3A indicate adsorption of 2.0×10¹⁴ antibody (binding to SEQ ID NO: 3) molecules per cm² to the CPE after only 10 minutes of incubation. This method of immuno-functionalization is due to the very strong physical attraction of the antibody molecules to the carbon surface and does not rely on multiple reaction steps, contrary to surface covalent immobilization strategies.

The strong bonds created between the antibody molecules (binding to SEQ ID NO: 3) and the CPE surface are highly stable, as the data in FIG. 6D suggest that less than 10% of the antibody molecules adsorbed to the surface desorbing after a period of 2 hours.

The functionalized CPE surface was analyzed by high-resolution SEM. The images, in FIGS. 6E-F, clearly show that the edge of an untreated μCF CPE surface was coated by an organic matter after antibody drop-casting.

In order to gather data regarding atomic content of the CPE before and after functionalization, Energy-Dispersive X-ray Spectroscopy (EDS) and X-ray Photoelectron Spectroscopy (XPS) analyses were performed, and the data are presented in FIGS. 6G-J and in Table 2 hereinbelow.

TABLE 2

| | | |
|---|---|---|
| | Untreated CPE | Immuno-functionalized |
| Element | (atomic content %) | CPE (atomic content %) |

EDS

| | | |
|---|---|---|
| C | 97.55 | 63.31 |
| O | 2.45 | 5.40 |
| N | — | 9.50 |
| Na | — | 10.78 |
| Cl | — | 7.43 |
| K | — | 2.43 |

XPS

| | | |
|---|---|---|
| C | 97.0 | 66.99 |
| O | 3.0 | 20.36 |
| N | — | 11.35 |
| S | — | 0.48 |
| P | — | 0.82 |

The results show an increase in the nitrogen content following adsorption of antibody molecules.

### Example 4

### Blocking of the Immuno-Functionalized Carbon Electrode

Salivary and nasal fluids contain many proteases tasked with aiding food disassembly and protection against infections. Proteomic analysis of human saliva has been recognized as a reliable non-invasive alternative to blood testing for diagnostics and disease monitoring [McDonald et al., J Dent Res 2011, 90, 268], including SARS-CoV-2 [M. Baghizadeh Fini, Oral Oncology 2020, 108, 104821]. However, the presence of highly active and concentrated proteases present in saliva, potentially hinders the proteomic salivary diagnostics [Thomadaki et al., J Dent Res 2011, 90, 1325], and is expected to affect 3CL^{pro} detection, although 3CL^{pro} (SEQ ID NO: 2) has no human homolog [Jin et al., 2020, supra]. These proteases could potentially cleave a selected 3CL^{pro} substrate (SEQ ID NO: 1), as the substrate is of high promiscuity. Therefore, there is a need for specific ‘fishing’ of 3CL^{pro} out of saliva.

As the advantage of a highly adsorbent large surface matrix (e.g., as demonstrated in FIG. 6D) may pose an even greater challenge for specific biosensors, a simple and rapid blocking step was conducted, using an agent that interferes with the interaction (e.g., adsorption) of biological species (e.g., proteins) with the electrode surface.

In order to prevent non-specific adsorption of the many species found in saliva, the immuno-functionalized CPE was soaked in bovine serum albumin (BSA; SEQ ID NO: 4) solution. BSA adsorbs to available open sites on the carbon electrode (which are not interacted with the selective antibody) and prevents non-specific adsorption of undesired components in the tested saliva samples. The effectiveness of this step is shown in FIG. 3B. Following bio-functionalization, the electrode is exposed to a saliva sample for two minutes; in this step, 3CL^{pro} (SEQ ID NO: 2) found in saliva samples of SARS-CoV-2-positive subjects specifically binds to the surface-embedded antibody molecule (binding to SEQ ID NO: 3).

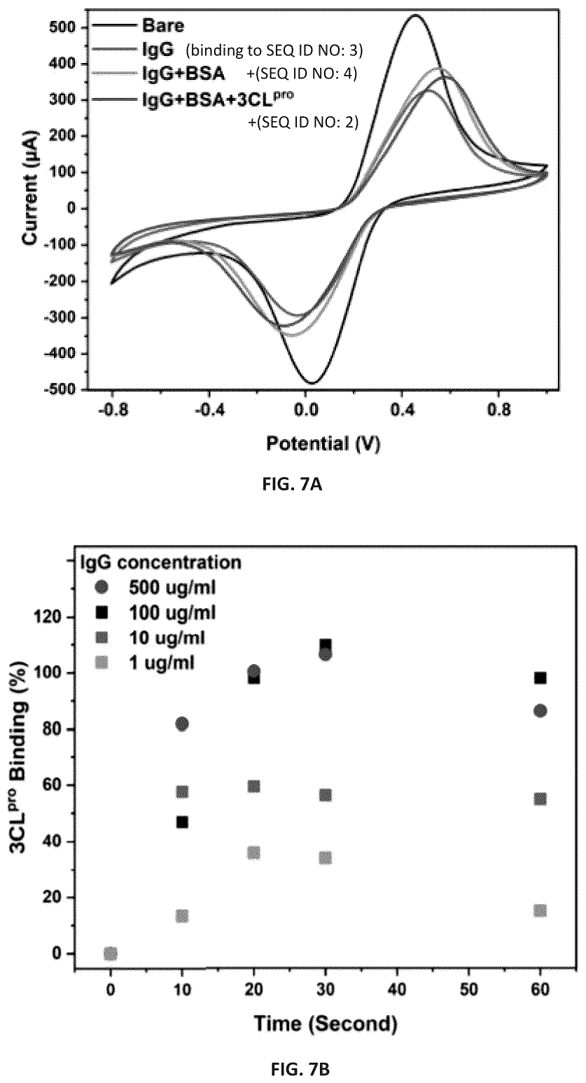

In order to examine the surface functionalizations, electrochemical CV measurements of [Fe(CN)₆]³⁻/[Fe(CN)₆]⁴⁻ were performed on CPEs in different steps: untreated CPE, immuno-functionalized CPE, the immuno-functionalized and blocked CPE, and the 3CL^{pro}-bound immuno-functionalized and blocked CPE and the results are presented in FIG. 7A. The data confirm the formation of the antibody recognition layer on the surface of the CPE, hindering electron transfer to the negatively charged RedOx agent [Patolsky et al. Anal. Chem. 71, 3171-3180 (1999)]. Additional CV measurements of the redox marker pBQ during CPE functionalization and blocking steps are presented in FIG. 3H.

The 3CL^{pro} specific-binding plot is shown in FIG. 3C, and indicates that a maximal binding is reached after only 30 seconds. Similar measurements for different densities of the 3CL^{pro}-specific antibody (binding to SEQ ID NO: 3) on the modified CPE surface are shown in FIG. 7B. Remarkably, maximal binding for the highest antibody surface density tested was reached after only 20 seconds of incubation of the surfaces with 3CL^{pro} (SEQ ID NO: 2).

These results open the possibility of shortening sample incubation time even further. Furthermore, the physical adsorption of the 3CL^{pro} IgG antibody (binding to SEQ ID NO: 3) to the high surface area of the CPE 3D matrix was shown to be highly efficient both in terms of kinetics (few minutes) and in retention of antibody affinity to 3CL^{pro} (SEQ ID NO: 2), as specific-binding of 3CL^{pro} is efficient even after antibody physical adsorption to the CPE.

### Example 5

### 3CL^{pro} Protein Detection

For 80 pmol of 3CL^{pro} (SEQ ID NO: 2), the measured pH change was 0.63 units, calculated to be about 0.74 units in a cell of 900 μl, which is expected to induce a peak shift of about 44 mV. Measurements correlate to this calculation nicely. CV results of healthy saliva ‘spiked’ with 58 pmol 3CL^{pro}, which were expected to produce a response of ΔE_{Peak}=˜36 mV, show an oxidation peak shift of 38 mV, as seen in FIG. 3D. FIG. 3D also indicates that enzyme-antibody binding does not affect the 3CL^{pro} enzymatic activity, as expected from an antibody (binding to SEQ ID NO: 3) that targets amino acids 81-132 in 3CL^{pro}, while the catalytic dyad is C145—H41 [Tahir ul Qamar et al., Journal of Pharmaceutical Analysis 2020, 10, 313]. Also, while SARS-CoV-2 3CL^{pro} (SEQ ID NO: 2) is considered highly conserved, sharing 96.08% sequence identity with SARS-CoV 3CL^{pro} and 87.00% with 3CL^{pro} from the middle east respiratory syndrome (SEQ ID NOs: 9 and 8, respectively), [Tahir ul Qamar et al., 2020, supra], sequence changes could be used to ensure antibody specificity.

After confirming the expected response in saliva containing 3CL^{pro} (SEQ ID NO: 2), non-specific adsorption of salivary proteases was tested by measuring the response to SARS-CoV-2 PCR-negative saliva. A non-specific response is not observed in CV measurements of saliva from healthy participants without added 3CL^{pro}, as shown in FIG. 3E, confirming specific ‘fishing’ of the 3CL^{pro} biomarker.

To examine the effect of pBQ presence in 3CL^{pro} (SEQ ID NO: 2) detection, a measurement of a solution containing the 3CL^{pro} with and without its substrate (SEQ ID NO: 1), using untreated CPE electrode, in the absence of pBQ was performed, and the results are presented in FIG. 2F. As can be seen, CV did not show any peaks, which does not allow for peak shift detection and thus demonstrate the need to include a pH-dependent redox probe in the electrolyte.

CV measurements were performed on healthy participants' saliva spiked with 3CL^{pro}, using CPE functionalized with myoglobin-specific antibody (SEQ ID NO: 5), that is non-specific to 3CL^{pro}. The results are presented in FIG. 3G, and show no response with ΔE_{Peak}=0 mV, further emphasizing the specificity of the immuno-functionalization.

CV measurements showing 3CL^{pro} activity positively detected in a saliva sample from PCR-positive to SARS-CoV-2 participants (25<Ct<31), as shown in FIG. 3F, is similar to CV results of saliva ‘spiked’ with 3CL^{pro} (SEQ ID NO: 2), shown in FIG. 3D. This confirms the real-world applicability of the herein exemplified methodology for SARS-CoV-2 detection, with a clear response of ΔE_{Peak}=28 mV indicating the 3CL^{pro} presence in the sample and enabling the detection of SARS-CoV-2 directly from untreated saliva samples.

To test the stability of 3CL^{pro} (SEQ ID NO: 2) in saliva, a sample from a healthy subject was spiked with 50 μg ml⁻¹ 3CL^{pro} and tested at different time points. The results are presented in FIG. 8A, and show that 3CL^{pro} is still active after 6 hours in saliva.

In addition, the kinetic stability of CPE immuno-functionalization was tested by storing immuno-functionalized CPEs under refrigeration, followed by testing 3CL^{pro} (SEQ ID NO: 2)-spiked saliva at different times. The results are presented in FIG. 8B and show that the immuno-functionalized CPE is still active after eight days of refrigeration.

As pH variability could be expected in broad screenings [Aframian et al. Oral Diseases 2006, 12, 420-423], samples of different initial salivary pH were tested and are presented in FIG. 8C. The data reveal that 3CL^{pro} (SEQ ID NO: 2) specific ‘fishing’ by antibody (binding to SEQ ID NO: 3) and electrode washing effectively prevents initial salivary pH from affecting peak shift results.

### Example 6

### Detection of SARS-CoV-2 in Clinical Samples

To validate the clinical detection of SARS-CoV-2 in comprehensive clinical samples, a set of twenty-four SARS-CoV-2 negative samples (i.e., healthy) and twenty-six SARS-CoV-2 positive samples (PCR-positive, 25<Ct<31) were tested.

Out of twenty-six SARS-CoV-2 positive samples, all have been positively detected and easily differentiated from healthy samples since the mean peak shift of SARS-CoV-2 positive samples is about 20 mV, as shown in FIG. 4A, while healthy samples' mean peak shift is about 0.35 mV. Results from patients indicate that SARS-CoV-2 positive samples contain 1-100 nM of 3CL^{pro} (SEQ ID NO: 2).

Significant oxidation-peak shift differences were also evident between measurements of samples from SARS-CoV-2 positive participants, and COVID-19 recovered patients, as summarized in FIG. 4B. Even with small sample size, these results confirm no lingering of 3CL^{pro} (SEQ ID NO: 2) activity after viral infection ceases.

Specificity and sensitivity values calculated relatively to PCR results are shown in Table 3, and a plot of peak shift as a function of 3CL^{pro} (SEQ ID NO: 2) concentration is shown in FIG. 9A.

TABLE 3

Peak shift detection

| | Positive | Negative |
|---|---|---|
| PCR-positive subjects (#) | 26 | 0 |
| PCR-negative subjects (#) | 0 | 24 |

| | |
|---|---|
| Specificity | 100% |
| Sensitivity | 100% |

The data on Table 3 demonstrate the successful performance of the detection platform, with 100% specificity and 100% sensitivity, LOD=6.6 μg ml⁻¹.

While targeting viral infection, 3CL^{pro} quantification was also demonstrated at concentrations ranging from 13 μg ml⁻¹ to 106 μg ml⁻¹, with R²=0.935. Taken together with the data on the tested subjects, these indicate that SARS-CoV-2 positive saliva samples contain 14-59 μg ml⁻¹ of 3CL^{pro} (SEQ ID NO: 2).

Infection kinetics of a single PCR-positive individual were measured using the detection platform for eight days starting from the onset of mild symptoms. The test results were compared to PCR Ct values and COVID-19 salivary antigen home detection kit results, and the results are presented in FIG. 9B, with respective photographs of the antigen home detection results presented in FIG. 9C. As can be seen, peak shift detection correlated with PCR Ct result, both showing undetectable values by Day 8 post-symptoms onset. COVID-19 salivary antigen home detection kit results were falsely negative for two days after PCR positive results, while the peak shift detection of SARS-CoV-2 gave false-negative results a day earlier than both of the other methods. These results indicate that the detection method is comparable with PCR detection.

3CL^{pro} originating from other coronaviruses (SARS-CoV and MERS-CoV; SEQ ID NOs: 9 and 8, respectively), human immunodeficiency virus (HIV) protease (SEQ ID NO: 10), and the human proteases chymotrypsin (SEQ ID NO: 12) and TMPRSS2 (SEQ ID NO: 7) were tested, and the results are presented in FIG. 10. As can be seen, no detectable responses were observed when measuring healthy saliva spiked with other proteases, as peak shifts were lower than the minimal detection limit of the novel detection platform.

Considering the high similarity shared between 3CL^{pro} from SARS-CoV, MERS-CoV and SARS-CoV-2 (SEQ ID NOs: 9, 8 and 2, respectively), these results are highly surprising and demonstrate the high specificity against potential interferents. These results are also highly reproducible and accurate, as shown in FIG. 4C, with ten consecutive experiments of any given saliva sample from SARS-CoV-2 negative subject gave near-identical results, with an oxidation peak shift standard deviation of 5 mV. The same holds for any given SARS-CoV-2 negative saliva sample tested multiple times.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An electrode having attached thereto an agent that specifically binds to a biomarker of a viral infection, wherein:
the biomarker is a proteolytic enzyme indicative of said viral infection; and/or
the biomarker is found in a saliva of a subject having said viral infection.

2. The electrode of claim 1, wherein said biomarker is said proteolytic enzyme.

3. The electrode of claim 2, wherein said agent that specifically binds to said biomarker is an antibody specific to said proteolytic enzyme.

4. The electrode of claim 1, wherein said biomarker is a SARS-CoV-2-specific proteolytic enzyme.

5. The electrode of claim 4, wherein said biomarker is found in a saliva of a subject having an active SARS-CoV-2 viral infection.

6. The electrode of claim 4, wherein said agent that specifically binds to said proteolytic enzyme is an antibody specific to said SARS-CoV-2-specific proteolytic enzyme.

7. The electrode of claim 4, wherein said SARS-CoV-2-specific proteolytic enzyme is 3CL^{pro} (SARS-CoV-2 3CL^{pro}).

8. The electrode of claim 7, wherein said 3CL^{pro} comprises an amino acid sequence as set forth in SEQ ID NO: 2.

9. The electrode of claim 1, being a carbon electrode, optionally a carbon fiber microelectrode.

10. An electrochemical system comprising the electrode of claim 1, the electrochemical system being configured such that when said viral biomarker is contacted with said electrode, a detectable change in an electrochemical parameter is generated.

11. The electrochemical system of claim 10, wherein said electrode forms a part of an electrochemical cell and the electrochemical cell is operable by electrically connecting said electrode to a power source.

12. The electrochemical system of claim 11, wherein the electrochemical cell is operable by contacting said electrode with an electrolyte.

13. The electrochemical system of claim 12, wherein said electrolyte comprises a substance that is capable of interacting with said biomarker, wherein a detectable change is an electrochemical parameter is generated in response to an interaction between said biomarker and said substance.

14. The electrochemical system of claim 13, wherein said electrolyte further comprises an electroactive agent that undergoes an electrochemically detectable reaction in response to said interaction, to thereby generate said change in said electrochemical parameter.

15. The electrochemical system of claim 14, wherein said biomarker, said substance and said electroactive agent are selected such that said interaction between said biomarker and said substance generates a moiety or species, and said electroactive agent undergoes an electrochemically detectable (e.g., redox) reaction in response to a presence of said chemical moiety or species.

16. The electrochemical system of claim 15, wherein said interaction between said biomarker and said substance results in a pH change and wherein said electroactive agent undergoes a pH-dependent electrochemically detectable reaction.

17. A method of determining a presence and/or amount of a viral biomarker in a sample, the method comprising contacting the sample with the electrode of claim 1, and determining a change in an electrochemical parameter generated upon operating an electrochemical system comprising the electrode of claim 1, wherein said change is indicative of the presence and/or amount of the viral biomarker in the sample.

18. The method of claim 17, wherein the sample is a biological sample drawn from a subject, the method being for determining a presence and/or amount of a viral infection in the subject.

19. The method of claim 18, wherein said biological sample is a saliva sample of the subject.

20. The method of claim 17, wherein said biomarker is SARS-CoC-2 3CL^{pro}, the method being of determining a presence and/or amount of a viral infection caused by SARS-CoV-2 in the subject.
